(19) United States
(12) Patent Application Publication
(10) Pub. No.: US 2011/0009332 A1
(43) Pub. Date: Jan. 13, 2011

(54) Therapeutic Treatment Of Wounds

(75) Inventor: Jorge Gianny ROSSINI, San Antonio, TX (US)

Correspondence Address: GROSSMAN, TUCKER, PERREAULT & PFLEGER, PLLC, 55 SOUTH COMMERICAL STREET, MANCHESTER, NH, 03101, US

(73) Assignee: SOUTHWEST RESEARCH INSTITUTE, San Antonio, TX (US)

(21) Appl. No.: 12/813,217

(22) Filed: Jun. 10, 2010

Related U.S. Application Data
(63) Continuation-in-part of application No. 12/500,196, filed on Jul. 9, 2009, now abandoned.

(51) Int. Cl. A61K 38/07; A61K 31/12; A61K 31/4965; A61K 38/06; A61P 17/02

(52) U.S. Cl. 514/18.6; 514/679; 514/255.06

(57) ABSTRACT

The present disclosure relates to compositions and methods for treating wounded skin and/or increasing the mechanical strength of wounded skin through the administration of an effective amount of a proteasome inhibitor to such wounded skin.

### CROSS-REFERENCE TO RELATED APPLICATIONS

The present application is a continuation-in-part of U.S. patent application Ser. No. 12/500,196, filed on Jul. 9, 2009, the teachings of which are incorporated herein by reference.

### STATEMENT REGARDING FEDERALLY SPONSORED RESEARCH OR DEVELOPMENT

This invention was made with United States Government support under United States Army Contract No. W81XWH-08-0189. The Government of the United States of America has certain rights in this invention.

### FIELD

The present invention relates to proteasome inhibitors and the use of an effective amount of such inhibitors for improving the properties of wounded skin during and/or after healing.

### BACKGROUND

The ability to heal by forming scars is essential for mammalian systems to survive wounding after injury. Normally, wound healing may be a relatively continuous process extending over a one-to-two-year period. The process may be conceptually divided into three fundamentally distinct stages. The first stage may be an intensely degradative phase called the inflammatory stage. It may occur immediately after injury and provide a means to remove the damaged tissues and foreign matter from the wound. Two-to-three days later, as fibroblasts from the surrounding tissue move into the wound, the repairing process may enter its second stage, the proliferation and matrix synthesis stage. The fibroblasts in the wound may proliferate and actively produce macromolecules, such as collagen and proteoglycans, which may be secreted into the extracellular matrix. The newly-synthesized collagen fibrils are cross-linked by lysyl oxidase and provide structural integrity to the wound. During this stage, fibroblasts also contract the intact collagen to reduce the surface area of the wound. This second stage may last about three weeks. In the final stage, the previous randomly-organized collagen fibril may then be aligned in the direction of mechanical tension and may become more organized so that the mechanical strength of the wound area can be increased. The repair process may be accomplished when the chemical and physical barrier functions of the skin are restored. Normal wound healing typically follows a relatively regulated course.

However, imbalances may cause abnormal and/or excessive scars to form. For example, if the biosynthetic phase continues longer than necessary or degradation of collagen decreases, hypertrophic scars may form. These scars may cause problems ranging from aesthetic deformity to severe limitation of motion. Hypertrophic scars may more frequently occur among children and adolescents, suggesting that growth factors may influence the development of this type of scar. Hypertrophic scars may be particularly common in patients who have burns or wounds that heal by secondary intention.

Another type of excess scar is the keloid. In this disorder, the cells appear to lack sensitivity to normal feedback signals. They may be larger than hypertrophic scars and may grow in an unregulated way, tending to invade normal tissue surrounding the wound. They rarely disappear spontaneously and may often recur after surgical excision.

Existing scar therapies may include surgery, mechanical pressure, steroids, x-ray irradiation, and cryotherapy. There are many disadvantages associated with each of these methods. Surgical removal of scar tissue is often incomplete and may result in the development of hypertrophic scars at the incision and suture points. The standard medicinal therapy is steroidal injections; these may be painful and often associated with unpredictable outcomes. X-ray therapy has been a predictably effective treatment to date; however, because of its potential for causing cancer, it is not generally recommended or accepted.

### SUMMARY

The present disclosure relates to compositions and methods for treating wounded skin to control scarring and/or increasing the mechanical strength of wounded skin through the administration of an effective amount of a proteasome inhibitor to such wounded skin.

### BRIEF DESCRIPTION OF DRAWINGS

The above-mentioned and other features of this disclosure, and the manner of attaining them, may become more apparent and better understood by reference to the following description of embodiments described herein taken in conjunction with the accompanying drawings, wherein:

FIG. 1 illustrates a diagram summarizing contemplated mechanisms of action of proteasome inhibitors in skin scarring after injury;

FIG. 2 illustrates a chart of proteasome inhibitor dosage versus epidermal thickness;

FIG. 3 illustrates cross-sections of epidermal skin growth illustrating differences in a) a control (untreated) skin sample and b) a treated skin sample at 10× and 40× magnification;

FIG. 4 illustrates cross-sections of epidermal skin growth illustrating differences in a) a control (untreated) skin sample and b) a treated skin sample at 10× and 40× magnification;

FIG. 5 illustrates cross-sections of epidermal skin growth illustrating differences in a a) a control (untreated) skin sample and b) a treated skin sample at 10× and 40× magnification;

FIG. 6 illustrates the effect of treatment with PS-1 on the tensile strength of rat skin after 28 days;

FIG. 7 illustrates the inhibition of 20S proteasome activity when treated with PS-1;

FIG. 8 illustrates the inhibition of 20S proteasome activity when treated with curcumin;

FIG. 9 illustrates the inhibition of 20S proteasome activity when treated with genestein;

FIG. 10 illustrates the effect on the activity of TGF-β1 upon the addition of PS-1;

FIG. 11 illustrates the effect on the activity of TGF-β1 activity upon the addition of PS-1;

FIG. 12 illustrates rat incisions for no dose, 1% PS1 dose and 5% PS-1 dose groups;

FIG. 13 illustrates tensile strength 7 days, 14 days, 28 days post-wounding in all groups;

FIG. 14 illustrates tensile strength 7 days post-wounding for no dose, low dose and high dose treated wounds;

FIG. 15 illustrates a comparison of tensile strength for untreated wounds and those treated with vehicle alone;

FIG. 16 illustrates the difference in tensile strength between no dose, low dose and high dose on day 28 post-wounding;

FIG. 17 illustrates the tensile strength of treated wounds and the corresponding vehicle controls of the treated wounds;

FIG. 18 illustrates the thickness of the treated and untreated wounds at 7 days, 14 days and 28 days;

FIG. 19 illustrates the wound thickness of adjacent DMSO controls;

FIG. 20 illustrates macrophage and lymphocyte accumulates over time in treated and untreated wounds; and

FIG. 21 illustrates the occurrence of epithelial hyperplasia over time in treated and untreated wounds.

FIG. 22 illustrates BMP-2 expression, normalized over 18S mRNA levels, in human dermal fibroblast cells after proteasome treatment with PS-1.

FIG. 23 illustrates BMP-2 expression, normalized over 18S mRNA levels, in human dermal fibroblast cells after proteasome treatment with curcumin.

FIG. 24 illustrates BMP-2 expression, normalized over 18S mRNA levels, in human dermal fibroblast cells after proteasome treatment with MG-132 and bortezomib.

FIG. 25 illustrates BMP-1 expression, normalized over 18S mRNA levels, in human dermal fibroblast cells after proteasome treatment with PS-1.

FIG. 26 illustrates BMP-1 expression, normalized over 18S mRNA levels, in human dermal fibroblast cells after proteasome treatment with curcumin.

FIG. 27 illustrates BMP-1 expression, normalized over 18S mRNA levels, in human dermal fibroblast cells after proteasome treatment with MG-132 and bortezomib.

FIG. 28 illustrates TGF-β1 expression, normalized over 18S mRNA levels, in human dermal fibroblast cells after proteasome treatment with PS-1.

FIG. 29 illustrates TGF-β1 expression, normalized over 18S mRNA levels, in human dermal fibroblast cells after proteasome treatment with curcumin.

FIG. 30 illustrates TGF-β1 expression, normalized over 18S mRNA levels, in human dermal fibroblast cells after proteasome treatment with MG-132 and bortezomib.

### DETAILED DESCRIPTION

The present disclosure relates to proteasome inhibitors and the use of proteasome inhibitors in the treatment of wounded skin. In addition, the present disclosure relates to a methodology for treating wounded skin and/or improving the mechanical properties of a subject's skin by, for example the administration of proteasome inhibitors in an effective amount. Wounded skin may be understood as skin challenged or otherwise damaged through disease mechanisms such as diabetic wounds, autoimmune disorders, aging, etc., or through external mechanisms such as burns, laceration, punctures, incisions, abrasions, etc. Treating or treatment may be understood as the use of the proteasome inhibitors to increase the mechanical properties of wounded skin during or after healing; such mechanical properties may include skin thickness and/or tensile strength. A subject may include mammalian or other animal species, such as human, feline, canine, bovine, porcine, rodent, ayes, etc.

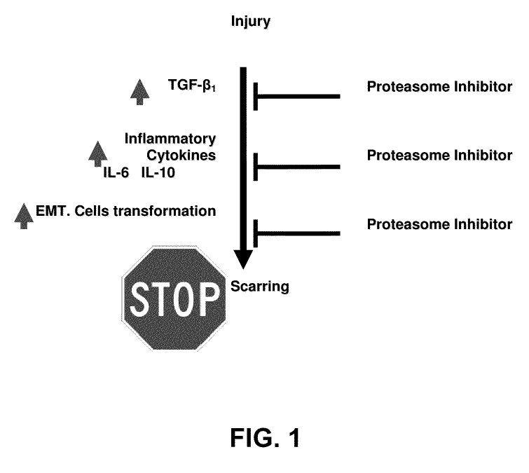

Without being bound to any particular theory, a number of biological mechanisms related to wound healing have been identified. For example, in scar formation, three biological mechanisms related to or regulating scarring have been identified. These three mechanisms may include growth factor regulation, inflammatory response, and transformation of differentiated epithelial cells into mesenchymal cells as illustrated in FIG. 1.

More specifically, it is believed that the presence, production or overproduction of transforming growth factor, TGF-β1, may play a role in the scar formation of adult wounds. For example, while TGF-β1 and TGF-β2 have been detected in neonatal and adult wounds, it has not been detected in fetal wounds, which may be repaired without any scar formation. In addition, it was found that fetal wounds healed with scar formation when exogenous TGF-β1 was added. Furthermore, it was found that TGF-131 knockout mice wounds healed with less granulation and relatively faster epithelialization when compare with control mice.

Inflammatory cytokines, interleukins 6 and 10, also have been found to be related to tissue scarring. IL-6 for example has been shown to be a relatively potent stimulator of fibroblast proliferation. IL-6 is diminished in fetal wounds but exogenous administration has been shown to lead to scarring. Accordingly, it is believed that inflammation may be detrimental to the wound-healing process. On the other hand, IL-10, a relatively potent anti-inflammatory cytokine, has been found to be elevated in fetal skin. In transgenic IL-10 knockout mice, skin healing occurs with scar formation.

A further factor, the transformation of differentiated epithelial cells and other local skin cells into non-differentiated, collagen producing mesenchymal cells, such as myofibroblasts, is also believed to be detrimental to the wound-healing process.

Myofibroblast accumulation at injury sites correlate with tissue scarring and fibrosis. The cells derive their name because they are like muscle like cells producing high level matrix protein, such as collagen-I and alpha smooth muscle actin (α-SMA), two markers of muscle cells.

These three processes may be blocked or inhibited by the use of proteasome inhibitors, which may, therefore, act as a single or multiple pathway healing mechanism regulating or reducing scar formation in favor of regeneration of the wounded skin. Proteasomes may be understood as molecules that may degrade unneeded or damaged proteins by proteolysis, breaking peptide bonds. Proteasomes regulate the concentration of proteins and degrade mis-folded proteins. Proteasome inhibitors may be understood as molecules that may block the action of proteasomes. The proteasome inhibitors may be relatively small molecules that may bind to the chymotryptic β₅ subunit of a proteasome leading to full inhibition of ubiquitinated protein hydrolysis.

The proteasome inhibitors may be naturally or synthetically derived. Examples of proteasome inhibitors may include, for example, aclacinomycin, apigenin, Z-Ile-Glu(OBu^{t})-Ala-Leu-H(PS-1), belactosin A, belactosin C, bortezomib, chrysin, cinnabaramide A, cinnabaramide C, cinnabaramide E, cinnabaramide G, curcumin, ECGC, eriodictyol, genistein, kaempferol, lactacystin, luteolin, MG132, naringenin, NPI-0052, omuralide, quercetin, salinosporamide A, TMC-95A, TMC-95B, TP-103, TP-104, TP-105, TP-106, TP-107, TP-108, TP-109, TP-110, TP-111, tryopeptin-A. An example of a bortezomib may be available under the tradename VELCADE from Millennium of Cambridge, Mass. Velvase, Velcade etc.

The proteasome inhibitors may directly or indirectly affect TGF-β1 activity. In one embodiment, proteasome inhibitors may reduce TGF-β1 expression, which may reduce fibrosis and/or scarring. In another embodiment, proteasome inhibitors may reduce BMP-1 expression. BMP-1, also known as PCP, is a protease enzyme which may degrade collagen and release TGF-β1 from the extracellular matrix. Reducing BMP-1 may, therefore lead to a reduction in the release of TGF-β1. In a further embodiment, proteasome inhibitors may increase BMP-2 expression. BMP-2 may antagonize the effects of TGF-β1 and, therefore, an increase in BMP-2 may reduce the effects of TGF-β1. It may be appreciated that a proteasome inhibitor may exhibit one or more of the above affects on TGF-β1, i.e., reducing TGF-β1 expression, reducing BMP-1 expression and/or increasing BMP-2 expression. Furthermore, in some embodiments, the proteasome inhibitors may positively affect some of the pathways, i.e., TGF-β1, BMP-1, BMP-2, yet negatively affect others. Thus, it is contemplated that, regardless of the pathway, proteasome inhibitors may be capable of reducing the activity of TGF-β1 overall.

In some examples, the proteasome inhibitors may exhibit a half maximal inhibitory concentration (IC₅₀) for a synthetic substrate for 20S proteasome in the range of 0.02 nM to 55,000 nM, including all values and increments therein, such as in the range of 0.02 nM to 10 nM, 400 nM to 600 nM, 8,000 nM to 12,000 nM, etc. The IC₅₀ may be understood as the amount of a particular substance/molecule needed to inhibit a given biological process in vitro by 50% and may be an indicator of the potency of a compound. The activity of the proteasome may be quantified by measuring the amount of fluorescence produced by cleavage of a fluorogenic substrate, such as LLVY-AMC, which when released by proteasomal cleavage, may emit fluorescent light.

The compositions described herein may be administered systematically or locally. Delivery may be topical (e.g., epicutaneous, creams, lotions, serums, etc.), parenteral (e.g., transdermal, transmucosal, intravenous, intraarterial, intramuscular, intradermal, subcutaneous, intraperitoneal, etc.), or enteral (e.g., oral or rectal). Topical administration may be made by the application of a carrier substance including the compositions described herein either directly or indirectly onto the surface of the skin. Intravenous administration may be made by a series of injections or by continuous infusion over an extended period. Administration may be performed at intervals ranging from one or more times a year, one or more times a month, one or more times a week, or one or more times a day. Administration may also be performed in a cyclical manner, that is, the administration may vary over a time frame and may be repeated, wherein portions of the time frame may include no administration or changing dosages. Treatment may generally continue until a desired outcome is achieved and/or to maintain such desired outcome.

The pharmaceutical formulation may include a compound of the present invention in combination with an acceptable carrier or vehicle. Acceptable carriers or vehicles may be understood as carriers or vehicles that may be understood as being relatively safe for exposure to mammals and/other other animal species. Such vehicles may, in some examples, include saline, buffered saline, 5% dextrose in water, dimethyl sulfoxide (DMSO), borate buffered saline containing trace metals, etc. Formulations may include one or more excipients, preservatives, solubilizers, buffering agents, albumin, lubricants, fillers, stabilizers, etc. Pharmaceutical compositions that may be used with the present invention may be in the form of sterile, non-sterile, non-pyrogenic, liquid solutions or suspensions, coated capsules, lyophilized powders, transdermal patches or other forms. Local administration may be facilitated by injection at a given site or insertion or attachment of a solid carrier at such site, or by direct topical application of a viscous liquid, or the like. Delivery may also be facilitated by controlled release compositions, including films, coatings, capsules, etc. Such controlled release compositions may be degradable, which may be understood as capable of being broken down in a given environment such as in saline solutions, oxygen, water, etc.; or biodegradable, which may be understood as capable of being broken down by enzymatic processes produced by living organisms.

The proteasome inhibitors may be administered in an effective amount, i.e., an amount which may produce a statistically significant effect. In some examples, an effective amount may include an amount that may increase BMP-2 expression, decrease BMP-1 expression and/or decrease TGF-131 expression. It is contemplated that in some embodiments, an effective amount may result in an increase in BMP-2 expression in a treated specimen over the BMP-2 expression of a control without treatment, wherein the increase in expression may be at least 150% or greater, such as in the range of 150% to 1,000%, including all values and increments therein. It is also contemplated that an effective amount of a proteasome inhibitor may cause a decrease in BMP-1 expression of a specimen treated with proteasome inhibitor as compared to a control without proteasome inhibitor treatment, wherein the BMP-1 expression of the treated specimen is 80% of the control expression or less, such as in the range of 10% to 80%, including all values and increments therein. Further, it is also contemplated that an effective amount of a proteasome inhibitor resulting in a decrease in TGF-β1 expression of a treated sample over a control sample would result in a TGF-β1 expression of 60% or less as compared to the control, such as in the range of 10% to 60%, including all values and increments therein.

For example, an effective amount of PS 1 may be 0.10 μM or greater, such as, for example 0.10 μM to 10.00 μM including all values and increments therein at 0.01 increments. An effective amount of curcumin may be, for example, 0.05 μM or greater, such as, for example, in the range of 0.05 μM to 100 μM including all values and increments therein at 0.01 increments. An effective amount of MG-132 may be, for example, 0.1 μM or greater, such as, for example, 0.1 μM to 1 μM, including all values and increments therein at 0.01 increments. An effective amount of bortezomib may be 0.1 μM or greater, such as, for example 0.1 μM to 1.0 M, including all values and increments therein at 0.1 M increments.

In some examples, an effective amount may include an amount that may improve mechanical properties of the skin. Mechanical properties may include, for example, tensile strength and, in one example, an effective amount may increase the tensile strength of wounded skin by 5% or greater, including all values and increments in the range of 5% to 100%, 5% to 50%, etc. after 28 days of healing as compared to wounded skin left untreated. It may therefore be appreciated that wounded skin without treatment may exhibit a first tensile strength after 28 days of healing T₁ and wounded skin with treatment may exhibit a second tensile strength after 28 days of healing T₂, wherein T₁*1.05≦T₂.

The proteasome inhibitors may, e.g., provide an increase in tensile strength such that after 28 days, the tensile strength may have a value of greater than or equal to 3.5 N/mm². More specifically, the tensile strength may be promoted to the range of 3.5 N/mm² to 5.0 N/mm² for a 28 day treatment protocol with the proteasome inhibitors and tested at a rate of 200 mm/minute with a 50 Newton load cell.

In other examples, administration of the proteasome inhibitors in an effective amount may also lead to the formation of a relatively thicker epidermal layer, as compared to the formation of epidermal layers without such inhibitors. For example, the epidermal layer treated with the proteasome inhibitors during scar formation may be 105% to 200% thicker than that of untreated epidermal layers 4 days after treatment, i.e., treatments may therefore increase the thickness by 5% to 100%, including all values and increments therein. It may be appreciated then that the epidermal layer of untreated wounded skin may exhibit a first thickness t₁ four days after treatment and the epidermal layer of treated wounded skin may exhibit a second thickness t₂ four days after treatment, wherein t₁*1.05≦t₂.

In addition, as alluded to above, the proteasome inhibitors may be administered in a given carrier, depending on the route of administration. In some examples, the carrier may include a solvent, such as protic solvents or polar protic solvents, including dimethyl sulfoxide (DMSO). However, it may be appreciated that other carriers may be utilized as well. The inhibitor may be administered at a concentration of 10 μm/mL or greater in such solvent solution, including all values and increments in the range of 10 μm/mL to 100 μm/mL. In addition, the dosage amount to, for example, a typical human may be in the range of 0.1 mg/kg to 1000 mg/kg, including all values and increments therein.

### EXAMPLES

The following examples are presented for illustrative purposes only and therefore are not meant to limit the scope of the disclosure and claimed subject matter attached herein.

### Example 1

Tissue samples were received and equilibrated for treatment. Media was retained for enzyme-linked immunosorbent assay (ELISA) using an EPIDERMFT kit, product number EFT-400. On the second day, PS-1 treatment was added to the samples, wherein the media was exchanged with compound in 4 ml. More specifically, three samples were maintained as control samples and included DMSO at a concentration of 62 μm/10 ml, with no added PS-1 treatment, 0.1 μM (0.620/10 ml) was added to three samples, 1 μM (0.620/10 ml) was added to three samples, and 10 μM (0.620/10 ml) was added to three samples.

The samples were left to incubate on days 3-5 and on day 6, the media was exchanged again. On the seventh day, tissue samples were removed and conditioned media was saved for ELISA. The samples were then measured using electron microscopy.

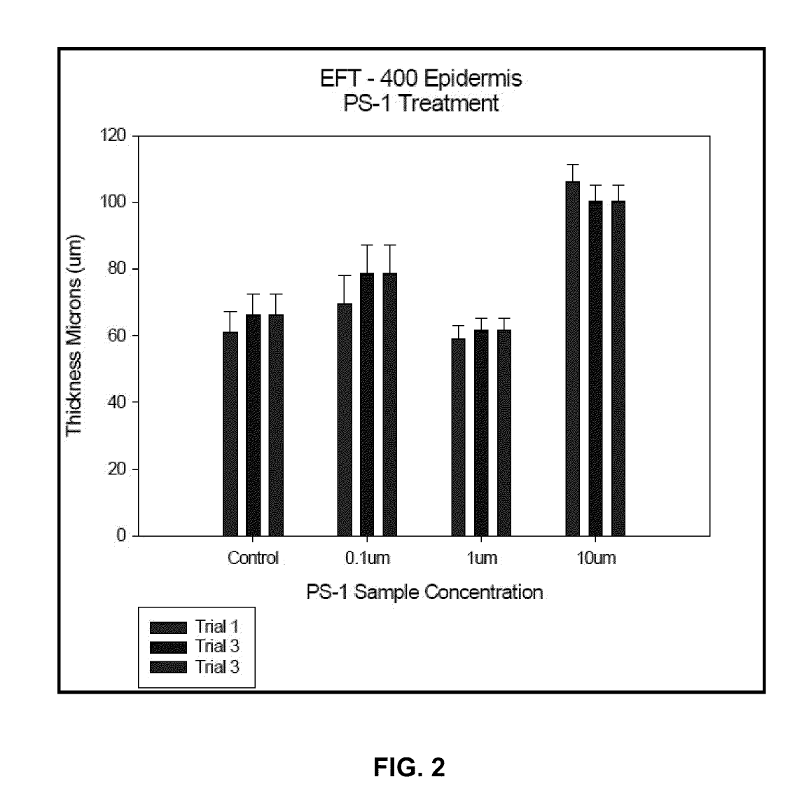

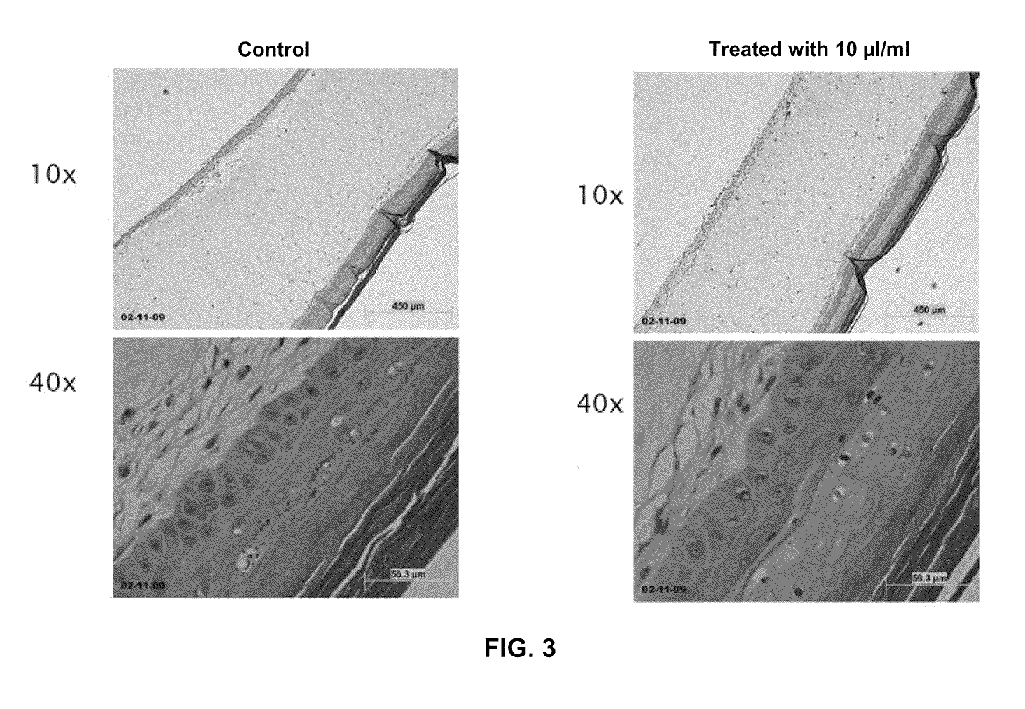

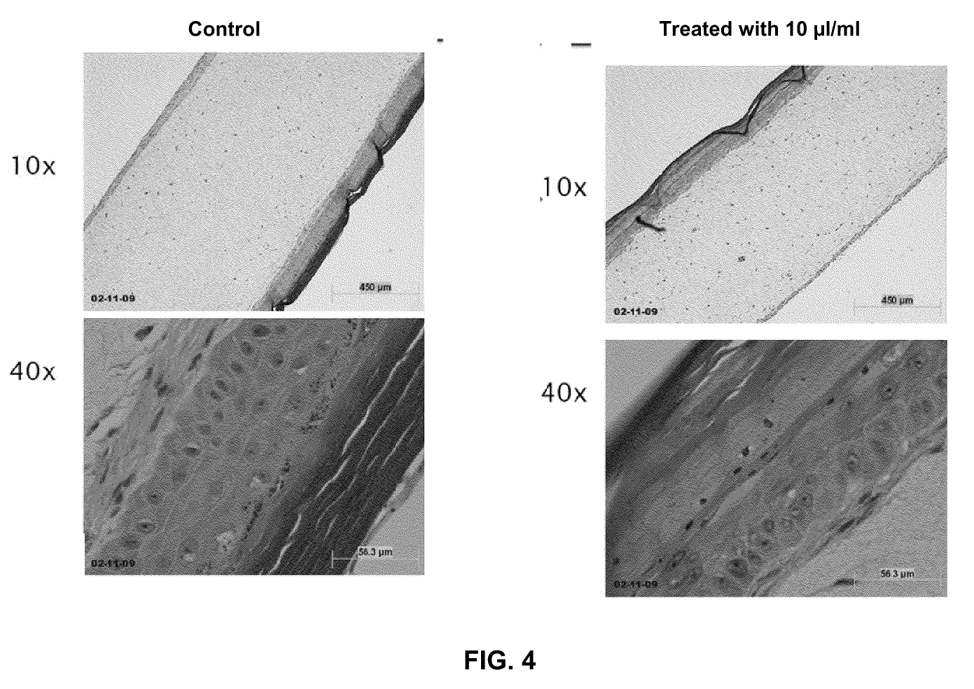

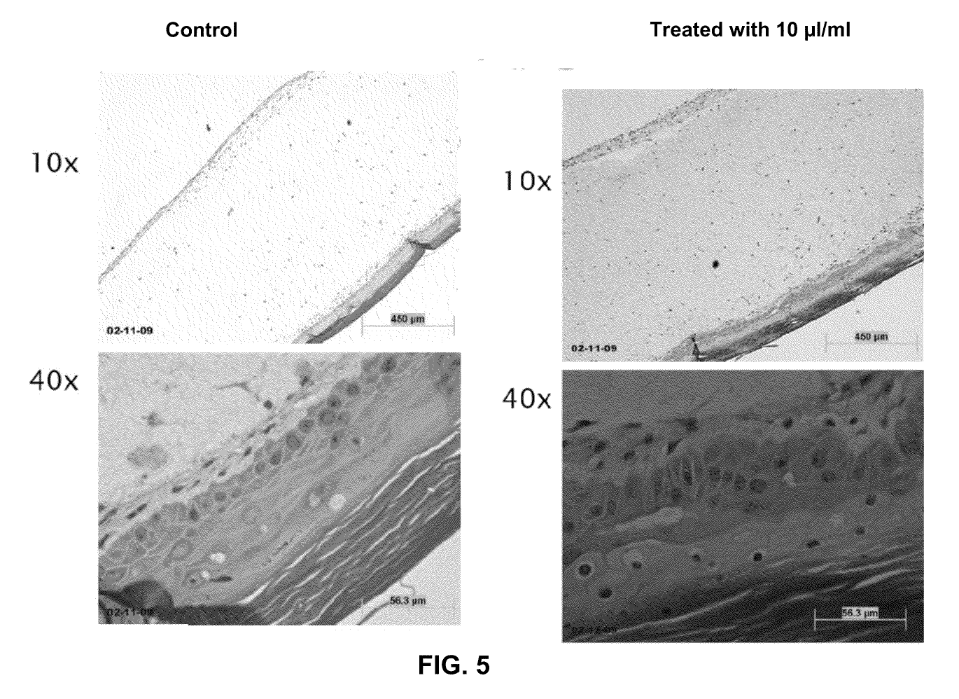

FIG. 2 illustrates changes in the epidermis thickness versus the amount of PS-1 added to the assays and the supporting data is presented in Table 1 below. As can be seen in the figure, the addition of 10 μm leads to a relatively significant increase in the thickness of the resultant epidermis in vitro compared with the other samples. FIGS. 3 through 5 illustrate the difference in thickness as between the control samples and the 10 μl/ml samples at 10× magnification and 40× magnification. As can be seen from these figures, the samples treated with PS-1 exhibited relatively well formed stratum granulosum as compared with the control samples.

TABLE 1
Comparison of Skin Thickness upon Addition of PS-1 at Varying
Concentrations

| EFT400- | Trial 1 | Trial 2 | Trial 3 | |
|---|---|---|---|---|
| Epidermis | (microns) | (microns) | (microns) | Error |
| Control | 61 | 73.73 | 66.22 | 6.39 |
| 0.1 μM | 69.47 | 61.85 | 78.82 | 8.49 |
| 1 μM | 59.13 | 54.09 | 61.6 | 3.82 |
| 10 μM | 106.31 | 96.2 | 100.16 | 5.09 |

In addition, upon review of the figures, it would appear that those samples treated with PS-1 resulted in relatively better developed stratum granulosum.

### Example 2

Testing of PS-1 was performed in rats and was provided at 1% and 5% in DMSO. The rats received 100 μl per wound site. The compound was administered daily for 5 days and skin was collected at 7 days, 14 days and 28 days. The biomechanical tensile strength of the skin was measured. Table 2 illustrates the testing data after 28 days for a control sample without treatment, 1% PS-1 in DMSO and 5% PS-1 in DMSO.

TABLE 2
Tensile Strength of Rat Skin After 28 Days of Treatment

| Sample | Control | 1% PS-1 in DMSO | 5% PS-1 in DMSO |
|---|---|---|---|
| 1 | 4.451418 | 3.707378 | 4.066714 |
| 2 | 2.425559 | 3.469754 | 4.378924 |
| 3 | 3.973553 | 3.655297 | 3.804534 |
| 4 | 2.332806 | 4.169227 | 5.041809 |
| 5 | 2.422365 | 4.475662 | 5.191631 |
| Mean | 3.12114 | 3.895464 | 4.496723 |
| StDev | 1.011167 | 0.414084 | 0.603723 |
| SEM | 0.452208 | 0.185184 | 0.269993 |

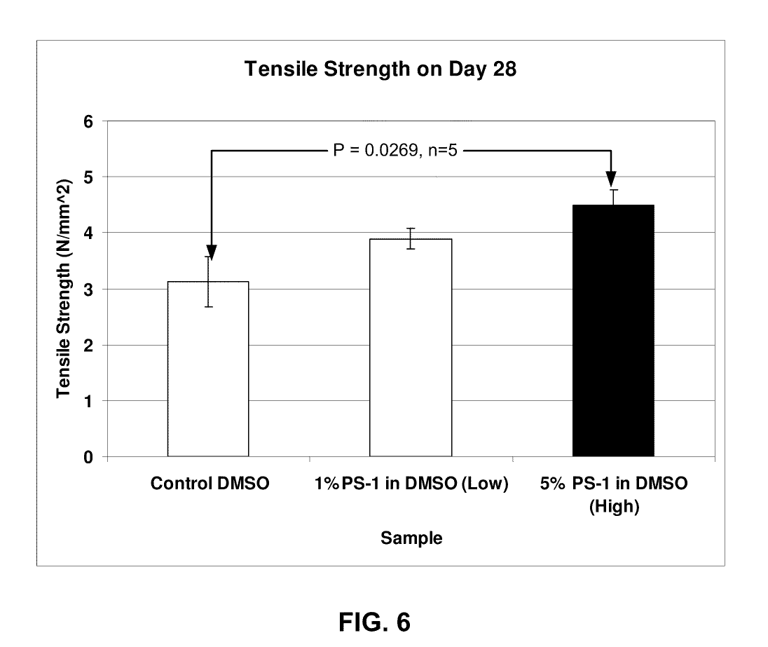

As illustrated in FIG. 6 and seen in the above table, the rat skin treated with 5% PS-1 in DMSO resulted in a relatively stronger tissue.

### Example 3

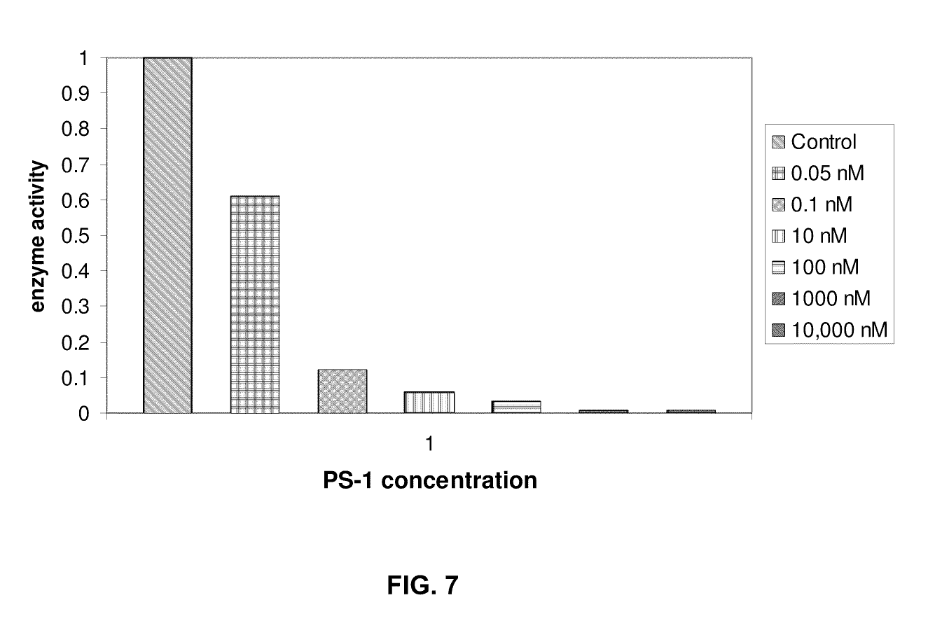

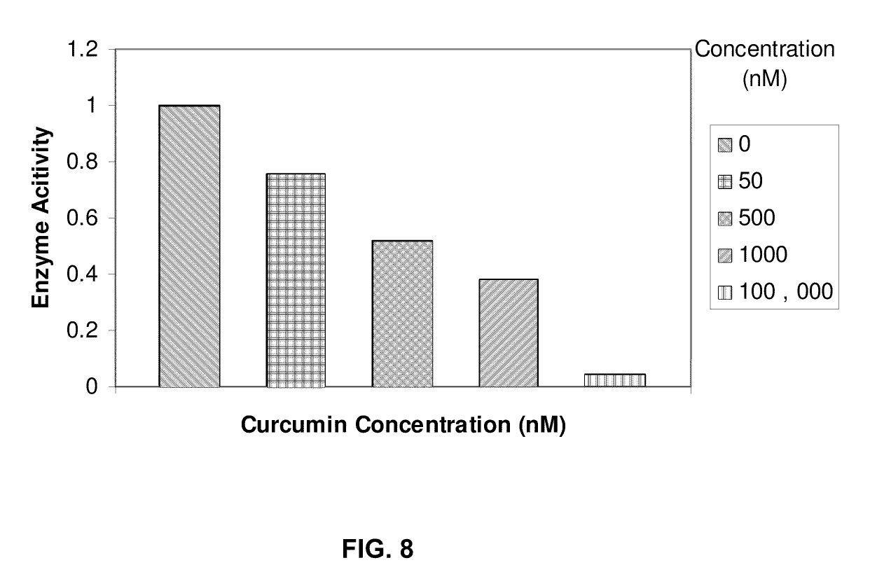

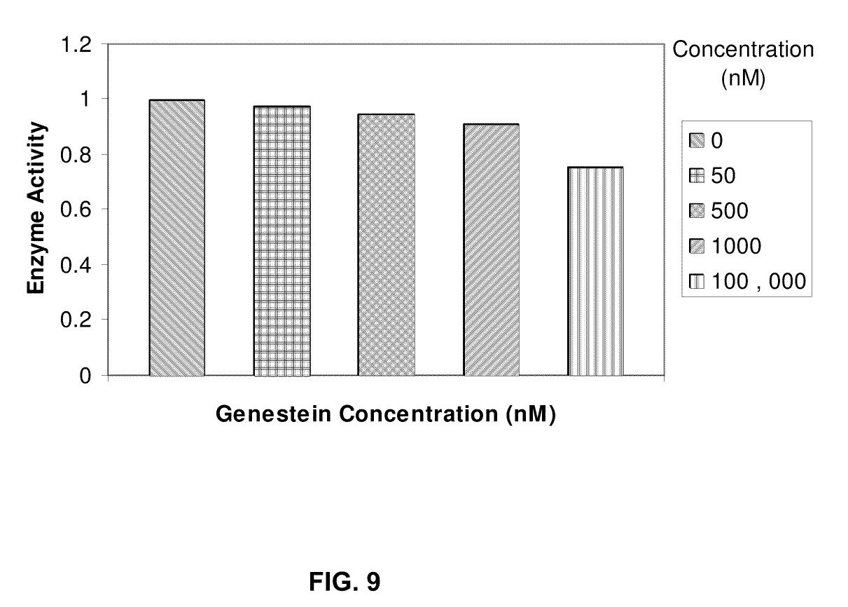

The enzyme activity of 20S proteasomes was measured upon increasing addition of the proteasome inhibitors PS-1, curcumin and genestein in concentrations ranging from 0 nM to 10,000 nM. In particular, FIG. 7 illustrates the inhibition of 20S proteasomes upon the addition of PS-1. As can be seen in the figure, as the concentration of the PS-1 increased, the enzyme activity decreased. FIG. 8 illustrates the inhibition of the 20S proteasomes upon the addition of curcumin. As can be seen in the figure, as the concentration of the curcumin increased, the enzyme activity of the proteasome is shown to decrease as well. FIG. 9 illustrates the inhibition of the 20S proteasomes upon the addition of genestein. As can be seen in the figure, there was a relatively smaller decrease in enzyme activity upon the addition of increasing concentrations of genestein.

### Example 4

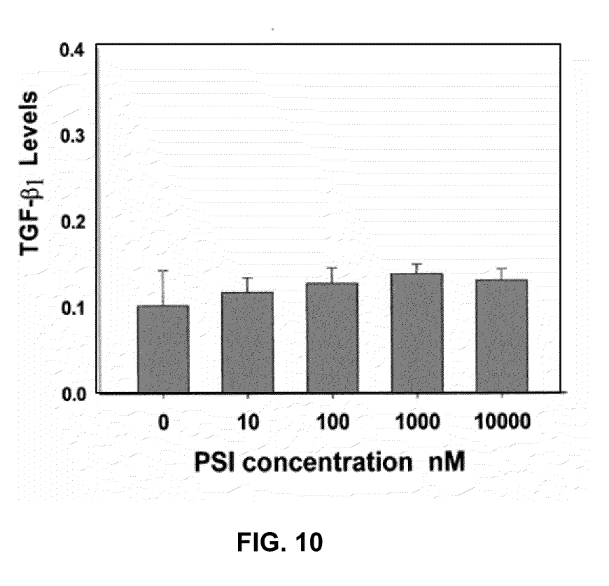

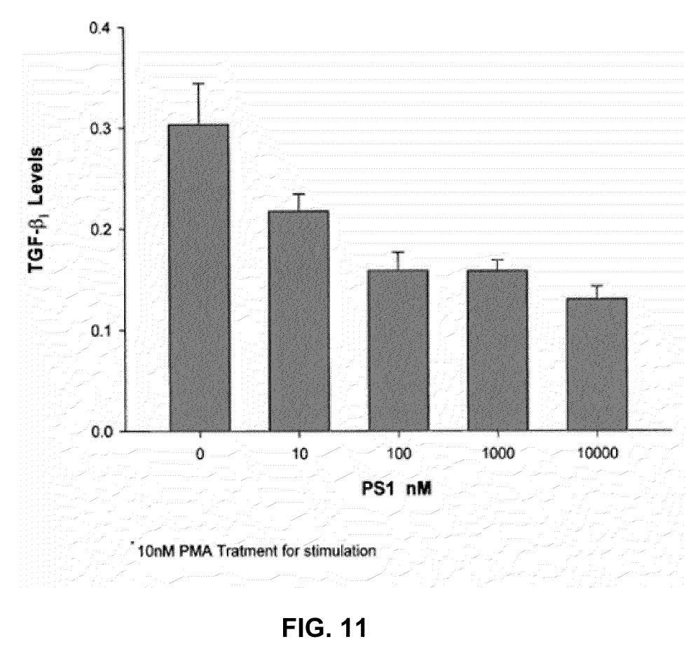

Cyto-toxicity analysis was performed to illustrate the inhibition of TGF-131 in human dermal fibroblasts. Both normal human dermal fibroblast cells (i.e., undamaged or non-wounded skin cells) and challenged (damaged) human dermal fibroblast cells were treated with concentrations of PS-1 ranging from 0 nM to 10000 nM and the effect of the various PS-1 concentrations on the normal and challenged cells was measured. It is noted that the challenged human dermal fibroblast cells were treated in vitro with 10 nM of phorbol 12-myristate 13-acetate (PMA) to simulate wounding. FIG. 10 illustrates that there was relatively little change in TGF-β1 protein expression with the addition of increasing concentrations of PS-1 in normal cells, indicating that the PS-1 does not appear to negatively affect the baseline levels of TGF-β1. FIG. 11 illustrates TGF-131 protein expression upon addition of PS-1 in cells challenged by the addition of the 10 nM of PMA. As can be seen in FIG. 11, increasing the concentration of PS-1 reduced TGF-β1 protein expression to similar levels seen in normal cells.

### Example 5

Using a standard incisional wound healing model, two 6 cm linear incisions were made through the panniculus carnosum on the dorsal aspect of 10 to 12 week old male Sprague-Dawley rats 1 centimeter on either side of midline running cephalid to caudal using a prefabricated template. Three surgical clips were used to close the incision and serve as guides for sectioning on harvest day.

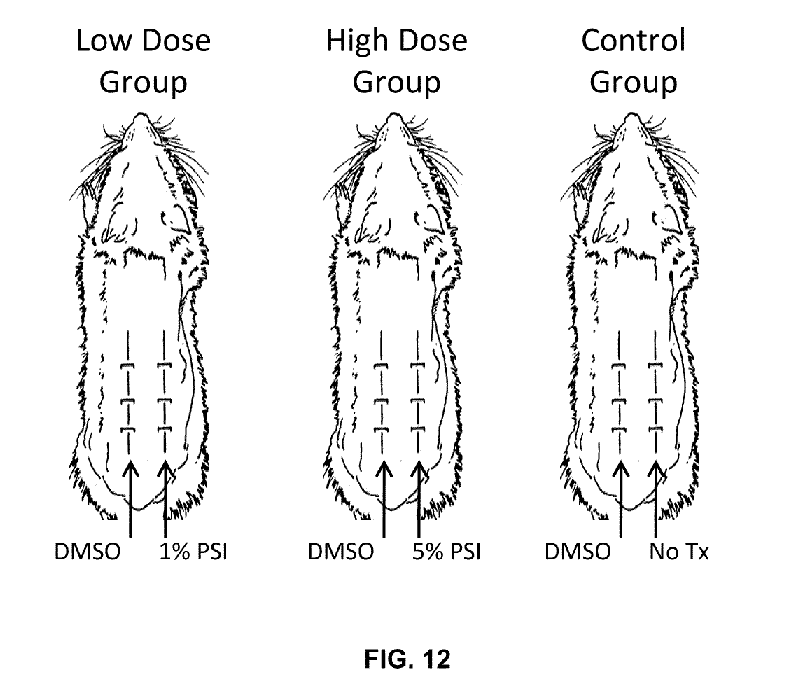

Rats were randomly assigned to 1 of 3 treatment groups, indentified by the treatment administered to the wound located on the left. All wounds located on the right, regardless of treatment group, received vehicle (100% DMSO) as a control. As illustrated in FIG. 12, Group 1 received Proteasome Inhibitor I (PS-1) at 1% w/w in DMSO; Group 2 received Proteasome Inhibitor I at 5% w/w in DMSO; and Group 3 remained untreated. Treatment was administered by applying 100 micro liters into (at time of closure) or onto the wound every 24 hours for the first 48 hours beginning at the time of wound closure, totaling 3 doses per wound.

On days 7, 14 and 28 post-wounding, the rats were euthanized and the entire dorsal skin excised. Four 8 mm sample strips were cut perpendicular to the wounds, to incorporate both the treated wound and the adjacent vehicle wound. The sample strips were then cut in half, equidistance from the two parallel wounds, yielding a total of four samples per wound, and yielding a compliment vehicle wound for each treated sample. One cephalic and one caudle sample were immediately prepared for histology, with the remaining two strips immediately prepared for tensometry.

Prior to sample loading for testing by tensometry, the underlying subcutaneous fascia was removed to the level of the panniculus carnosum using sharp dissection, following a natural dissection plane. For the day 28 wounds, the 8 mm samples were divided into 4 mm strips prior to loading secondary to slippage issues encountered during the pilot study. Samples were loaded into a Lloyd Instrument materials testing system (model LRX plus, Fareham, Hampshire, UK) with a 50 N load cell, with the wounds perpendicular to the grips and pulled at a rate of 200 mm/min. Wound thickness, just superficial to epithelium and just deep to panniculus carnosum, was measured at the time of testing with digital calipers (Absolute Digimatic Caliper, Mitutoyo, Kawasaki, Kanagawa, Japan). The wound width was measured at a preload of 0.5N by obtaining the correlating still frame image from a hands free mounted video camera (HDR-SR1 Handycam® camcorder, Sony, San Diego, Calif.). These two measurements were then used to calculate the cross sectional area of the wounds and the subsequent tensile strength (N/mm²).

Histological samples were fixed in 10% formalin, routinely processed, embedded in paraffin, and sectioned at approximately 5 μm. Cross sections of skin were stained with hematoxylin and eosin (H&E) and Masson's Trichrome using routine methods. All slides were subjectively evaluated on an Olympus BX40 microscope (Olympus America, Center Valley, Pa.) by a board-certified veterinary pathologist blinded to the treatment groups. The following parameters were determined for each sample: (1) collagen density (0=no mature collagen, 1=loose, 2=intermediate, 3=dense); (2) collagen maturity (1=immature, 2=intermediate, 3=mature); (3) amount and type of inflammation present in the dermis (0=none, 1=minimal, 2=mild, 3=moderate, 4=severe); (4) epithelial hyperplasia (0=none, 1=minimal, 2=mild, 3=moderate, 4=severe); (5) amount of epidermal necrosis over wound (0=0%, 1=25%, 2=50%, 3=75%, 4=100). Images were captured using an Olympus BX41 microscope (Olympus America, Center Valley, Pa.) and an Olympus DP71 digital camera (Olympus America, Center Valley, Pa.). Captured images of Masson's Trichrome sections under a 4× objective were uploaded into ImageJ (NIH) in order to calculate the cross sectional area of immature collagen within day 28 wounds. For statistical purposes, samples were averaged to provide a single score in each parameter per wound.

A paired student's t-test was used for statistical comparisons between adjacent wounds. A tukey's comparison was used for statistical comparison of wounds between different rats. A Kruskal-Wallis test was used for comparing graded histology. A Chi square was used to determine statistical meaning of epithelial necrosis.

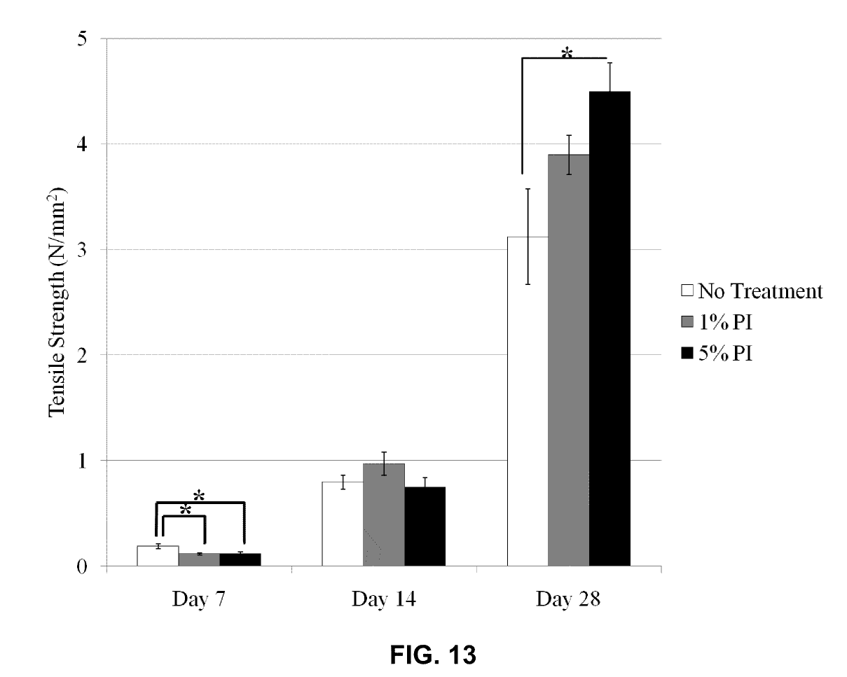

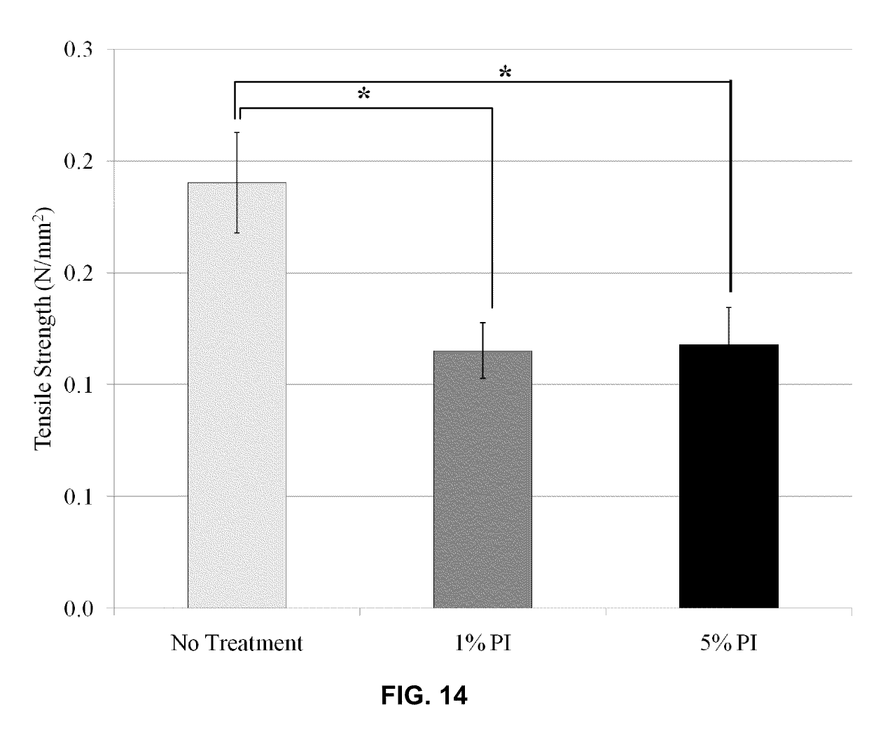

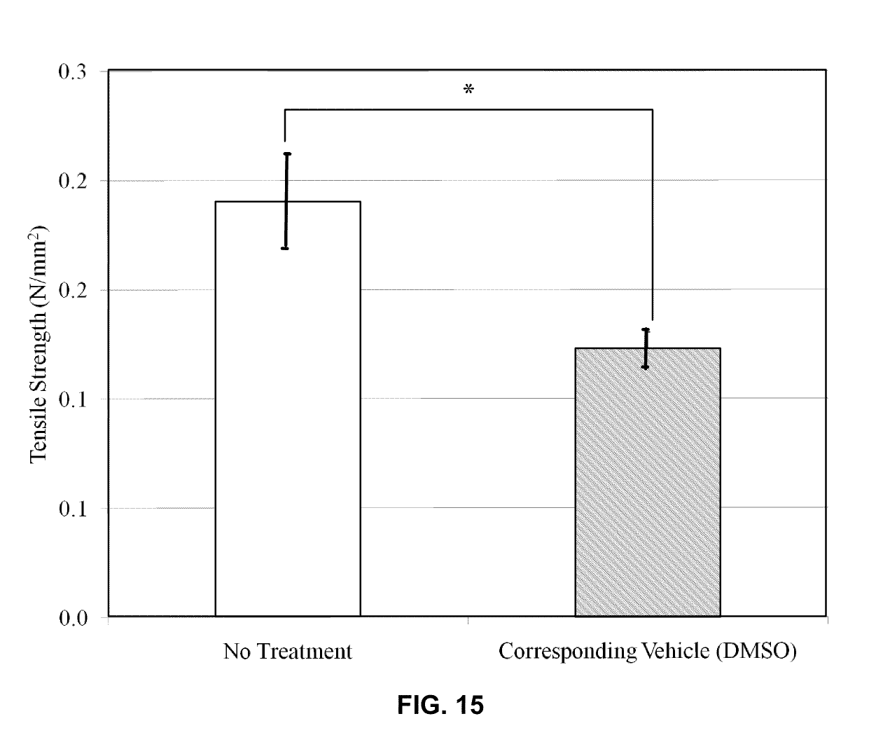

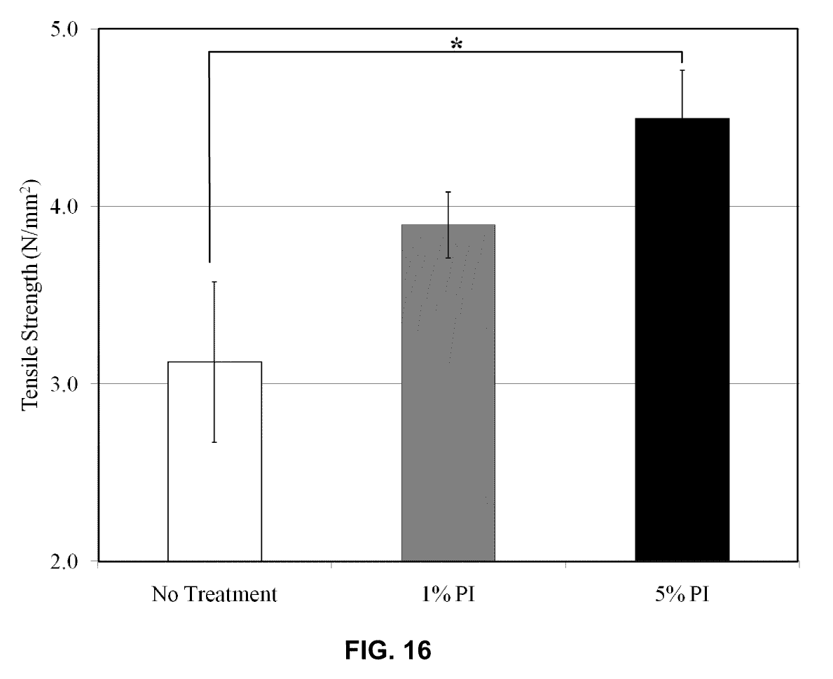

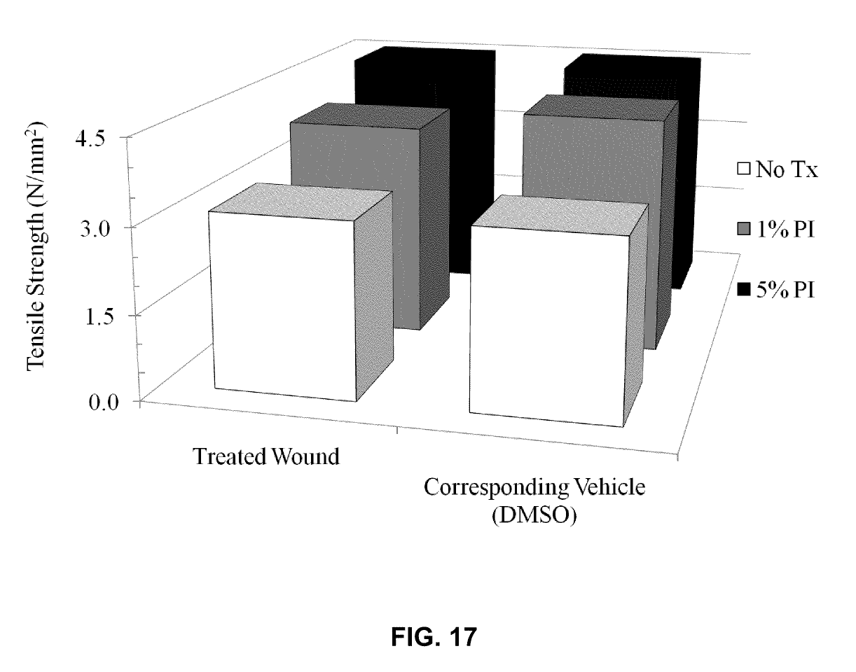

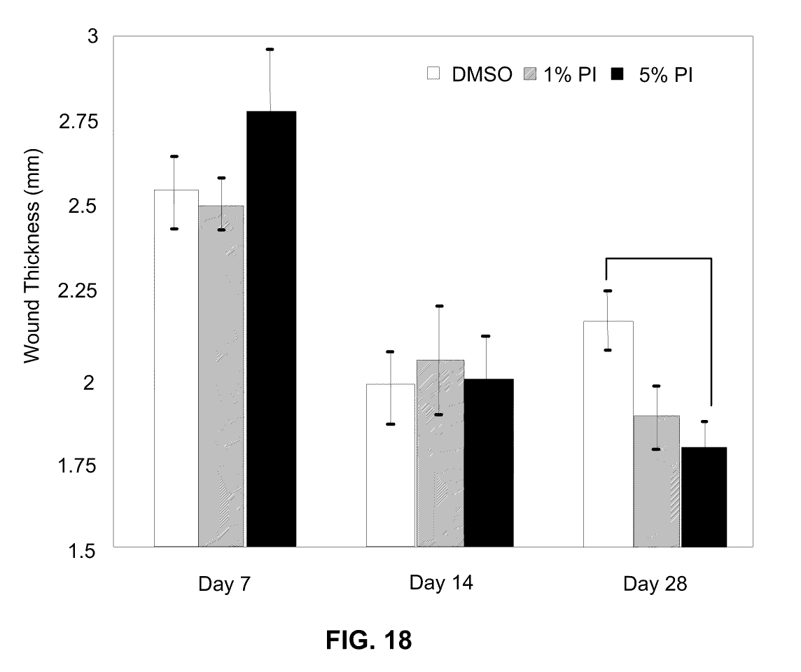

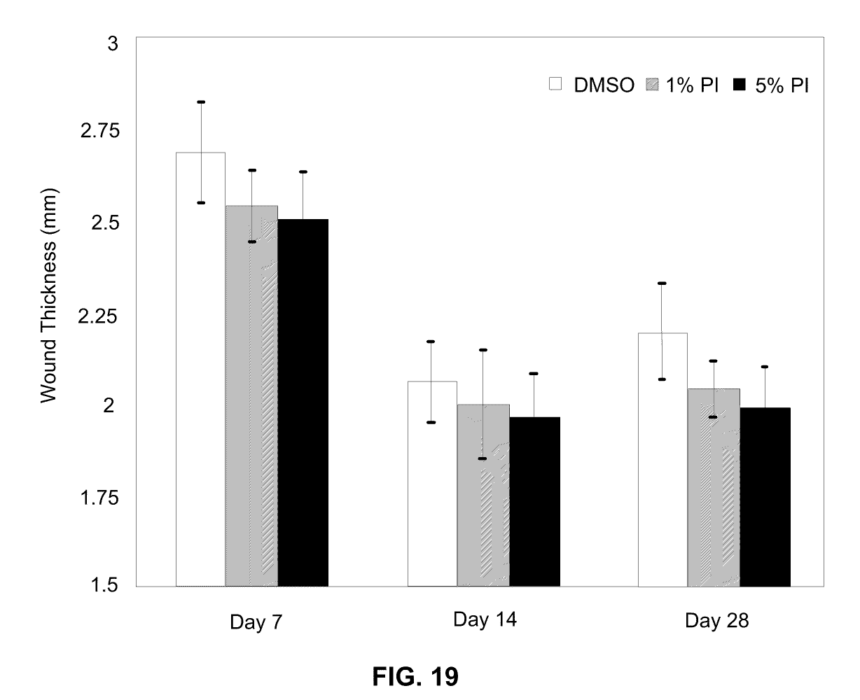

It was found that tensile strength increased with time post-wounding in all groups, as illustrated in FIG. 13. At day 7, tensile strengths were significantly lower for low dose and high dose treated wounds compared to the untreated wounds (p=0.0121 and p=0.0223, respectively), as seen in FIG. 14. However, as illustrated in FIG. 15, the untreated wounds were significantly stronger than all wounds receiving vehicle alone, including adjacent wounds on the control rats that received DMSO (p=0.0212). By day 14, no significant differences in tensile strength existed between any wounds. On day 28, as illustrated in FIG. 16, a dose dependent trend was appreciated, with the wounds receiving 5% PI (proteasome inhibitor) having significantly increased tensile strength over the untreated wounds (p=0.0269). Despite this dose dependent trend, no significant difference existed for the 1% PI wounds. No significant differences existed between the wounds directly treated with proteasome inhibitor and their adjacent wounds that received only DMSO vehicle. Secondary to the vehicle wounds behaving like their adjacent PI treated wounds, a similar PI dose dependent trend emerged among the vehicle controls, though failing to reach significant differences, as illustrated in FIG. 17. Wound thickness, as measured from epithelium to just beneath panniculus carnosum, decreased from day 7 to day 14 for all wounds. Then from day 14 to day 28, wound thickness slightly increased for control wounds, while PI treated wounds continued to decrease in thickness. By day 28, 5% PI wounds became significantly thinner than the untreated control wounds (p=0.013), meanwhile, 1% PI wounds were thinner than untreated control wounds, but just lacked significance (p=0.057), as illustrated in FIG. 18. A similar response existed for the adjacent DMSO controls FIG. 19. In these in vivo measurements, the skin thickness was measured without distinguishing the dermis or epidermis. Furthermore, upon incision, inflammation occurred subsequently resulting in thicker skin overall. After PS-1 treatment, the wound thickness normalized faster and with relatively less signs of immature collagen.

More specifically, histology revealed time dependent changes of dermal healing, beginning with granulation tissue seen on day 7, followed by primarily disorganized collagen on day 14, and ending with basket weaved collagen consisting of fibrils thinner than collagen in non-wounded adjacent skin by day 28. Histology graded in a blinded fashion revealed no differences in the collagen progression between control, vehicle or treated wounds. Differences in overall collagen appearance were found among day 28 wounds, with wounds belonging to low dose or high dose treated rats, independent of direct administration to the wound, receiving higher scores over control wounds, this could not be statistically confirmed as demonstrated in Table 3.

TABLE 3
Overall Dermal Collagen Response

| | Day 7 | Day 14 | Day 28 |
|---|---|---|---|
| Control Treated | 0.8 ± 0.3 | 1.9 ± 0.3 | 2.6 ± 0.5 |
| Corresponding Vehicle | 0.8 ± 0.3 | 2.0 ± 0.0 | 2.6 ± 0.5 |
| PS-1 1% Treated | 0.8 ± 0.3 | 2.1 ± 0.3 | 2.9 ± 0.3 |
| Corresponding Vehicle | 0.8 ± 0.3 | 2.1 ± 0.3 | 2.9 ± 0.3 |
| PS-1 5% Treated | 0.7 ± 0.3 | 2.0 ± 0.0 | 2.8 ± 0.3 |
| Corresponding Vehicle | 0.7 ± 0.3 | 2.1 ± 0.3 | 3.0 ± 0.0 |

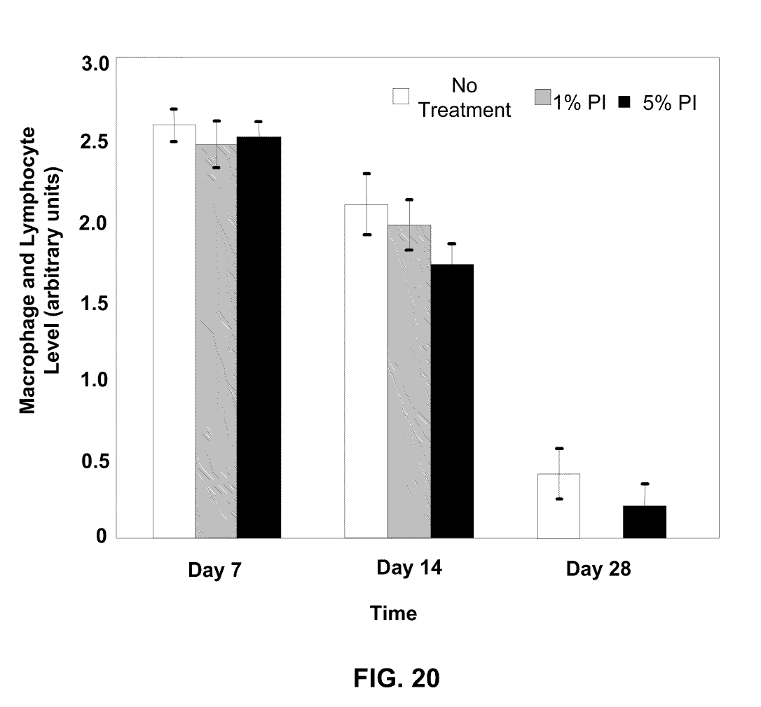

Inflammatory response, as measured by cellular infiltration of the dermis by neutrophils (acute), macrophages and lymphocytes (chronic) was most pronounced on day 7 for all three cell types. By day 14, the accumulation of neutrophils was nearly extinct for vehicle and treated wounds, most notable in the rats receiving 1% PI, while wounds receiving no direct intervention continued to have a moderate response. Concurrently, macrophage and lymphocyte accumulation declined by approximately one third and one half, respectively, with all wounds responding similarly. On day 28, only two samples, one from a non-treated wound and one from a 5% PI wound, contained visible dermal neutrophils. The section taken from the 5% PI wound was noted to have a gross infection. As measured through visual observation, macrophage and lymphocyte accumulates continued to decline, most notably in the low dose rat wounds, but persisted for all wounds as illustrated in FIG. 20.

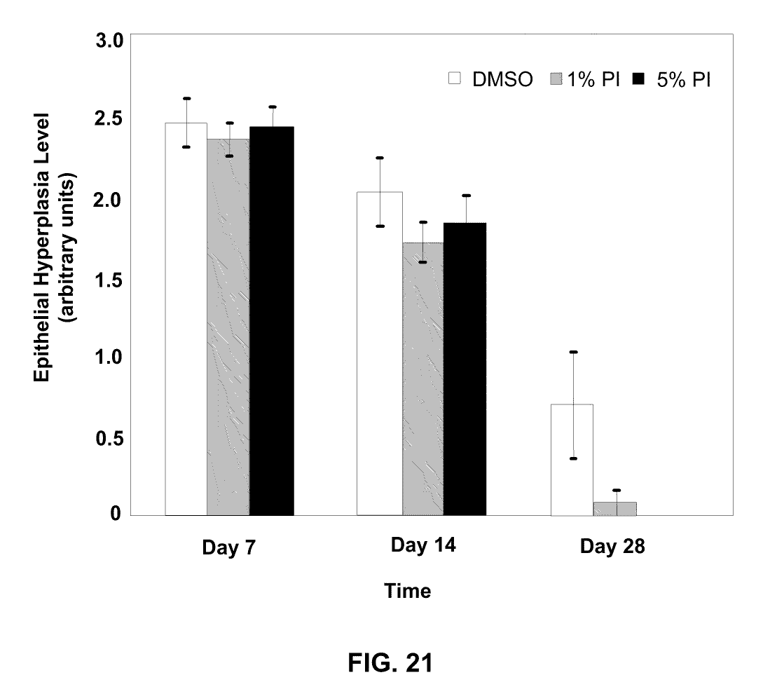

Epidermal hyperplasia took on a pattern that appeared independent of DMSO, but rather dependent upon PI administration. This histology find was most distinct when grouping wounds according to rat group rather than individual wound group. Epithelial hyperplasia was most pronounced at day 7 for all groups, decreasing in all groups by day 14, and becoming minimal to no longer present for most wounds by day 28. As measured by visual observation, wounds belonging to non-PI treated rats were more likely to present with some degree of epithelial hyperplasia, 60% of wounds in control rats versus 8% of wounds in 1% PI rats and 20% of wounds in 5% PI on day 28 as illustrated in FIG. 21.

Lastly, histology revealed wounds receiving direct administration of the PI contained a foreign body reaction in the subcutaneous tissue, just beneath the panniculus carnosum, with the number wounds presenting with the reaction and the size of the reaction seemingly dependent upon the dose received.

### Example 6

An examination of gene expression of BMP-2 was conducted using a number of proteasome inhibitors including, proteasome inhibitor-1, curcumin, MG-132 and bortezomib. Human neonatal dermal fibroblasts (AllCells, Catalog No. HN006001) were maintained in Dulbecco's modified Eagle's medium (DMEM) supplemented with 10% fetal bovine serum. Early passage cells were plated at 2×10⁴ cells/cm² and grown in humidified 5% CO₂ at 37° C. for 24 hours. Spent media was replaced the following day with assay media including Proteasome inhibitor-1 was added in DMEM at 1 μM and 0.1 μM. Untreated cells were used as the control group. For the human osteogenesis PCR array, the experimental group was treated only at 1 μM.

Phenol-chloroform extraction was performed to isolate the total RNA for real time polymerase chain reaction array. More specifically, the total RNA was prepared using TRI REAGENT (Ambion, product number 9738), wherein the total RNA was obtained from the human neonatal dermal fibroblasts. Note that isolation was performed as per the instructions provided with the TRI REAGENT. The RNA was used for subsequent quantitative polymerase chain reaction (qPRC) gene expression of BMP-2 measured using hydrolysis probes (TAQMAN probes available from Applied Biosystems) as described further below.

Concentration and integrity of the obtained total RNA was assessed by absorbance in a spectrophotometer at 260 nm and 280 nm and by electrophoresis in 1% agarose with formaldehyde loading dye. For TAQMAN gene expression assays, cDNA was synthesized from 2 μg of total RNA using the High Capacity cDNA Reverse Transcription Kit (Applied Biosystems, product number 4368814), again following the protocol supplied with the kit. The resulting cDNA was diluted 1:10.

For TAQMAN gene expression, gene-specific fluorescent labeled TAQMAN primers for FAM-BMP2 (Applied Biosystems, product number Hs00154192.m1) and TAQMAN reference VIC-18S ribosomal RNA (Applied Biosystems, product number 4310893E) were used to target the gene of interest. A cocktail of universal master mix (Applied Biosystems, product number 4369016) was prepared individually for both set of primers which contained Taq Polymerase, dNTPs, and buffer. Template cDNA's were added separately to respective individual wells in triplicate samples. PCR was performed on StepOne Plus Real Time System instrument (Applied Biosystems). The amplification program consisted of 1 cycle of 95° C. with 10 minute hold (hot start) followed by 50 cycles of 95° C. with 15 second annealing hold and 1 minute 60° C. specified acquisition hold.

For human osteogenisis PCR array, total RNA was prepared using silica matrix purification using RNEASY MINI KIT (Qiagen, product number 74101) according to the manufacturer's recommendations. cDNA was synthesized from 1 μg of purified DNA-free RNA accordingly to RT²First Strand Kit (SABiosciences, C-03). The resulting cDNA was diluted 1:30 and amplified. The procedure for the PCR array using real-time PCR is based on the RT² Profiler PCR array system instructions for Human Osteogenesis (SABiosciences, product number PAHS-026C). A cocktail reaction mix was setup as recommended by the PCR manufacturer (SABiosceiences, product number PA-012). PCR was performed using the same temperature parameters as used with the StepOne Plus Real Time System instrument. The Ct value of endogenous reference gene (i.e. 18s) was used to control for input RNA and then used to normalize target gene (i.e. BMP-2) tested from the same cDNA sample (Δ Ct), and then calibrated to an internal reference sample (ΔΔCt). Change in gene expression was determined by the expression 2̂-(ΔΔCt).

In addition, as alluded to above a similar methodology using 10 μmM and 100 μM of curcumin, 0.1 μM and 1 μM of bortezomib, and 0.1 μM and 1 μM of MG-132 proteasome inhibitors was performed.

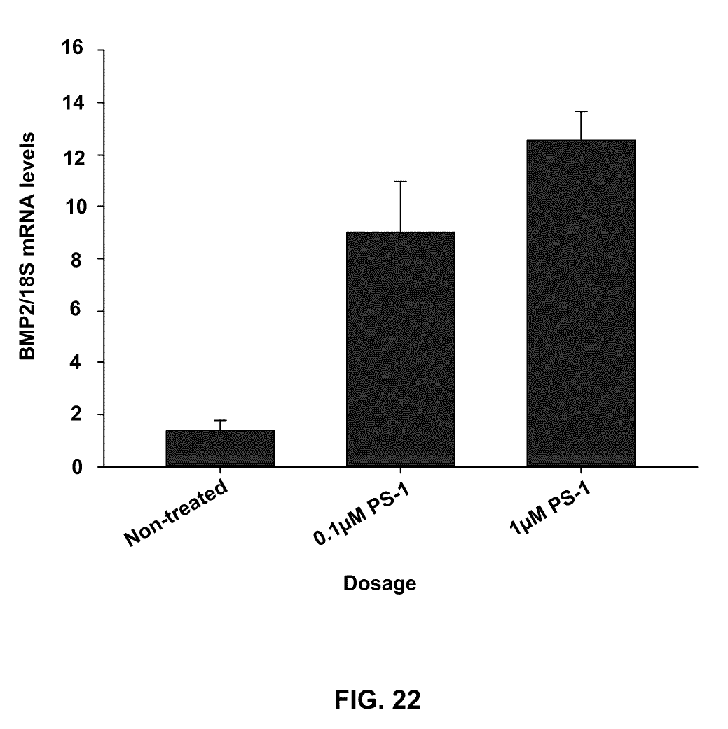

FIG. 22 illustrates BMP-2/185 mRNA levels in human dermal fibroblast cells after proteasome inhibitor treatment with 0.1 μM of PS-1 and 1 μm PS-1 as compared to the non-treated control. As seen in the figure, the levels of BMP-2 increase upon treatment with the PS-1 inhibitor. For example, with treatment of 0.1 μm of PS-1, the BMP-2 levels increased from approximately 1.38 au (arbitrary units) to 9.01 au and with treatment of 1.0 μm, the BMP-2 levels increased from approximately 1.38 au to 12.5 au.

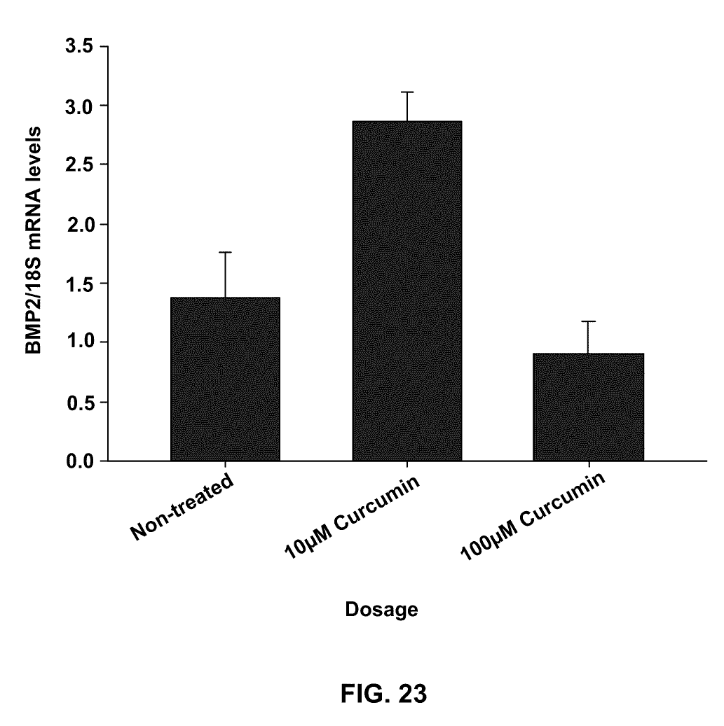

FIG. 23 illustrates BMP-2/18S mRNA levels in human dermal fibroblast cells after proteasome inhibitor treatment with 10 μM of curcumin and 100 μm of curcumin as compared to the non-treated control. As seen in the figure, the levels of BMP-2 increase upon treatment with 10 μM of the curcumin inhibitor from approximately 1.38 a.u. in the non-treated sample to 2.87 a.u. in the treated sample. In the sample treated with 100 μM of curcumin, the level of BMP-2 decreased from approximately 1.38 a.u. to 0.903 a.u.

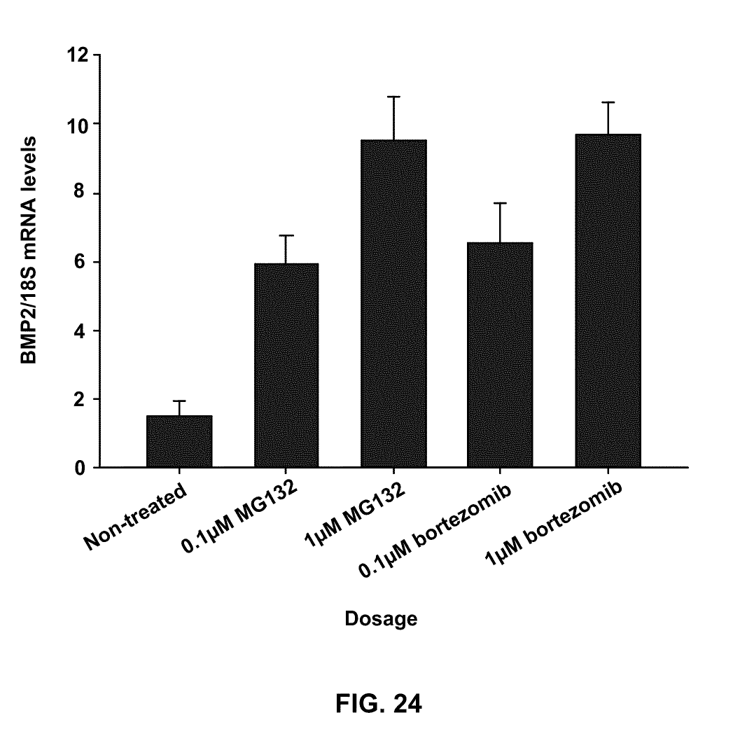

FIG. 24 illustrates BMP-2/18S mRNA levels in human dermal fibroblast cells after proteasome inhibitor treatment with 0.1 μM of MG132 and 1 μM of MG132 as compared to the non-treated control as well as treatment with 0.1 μM of bortezomib and 1 μM of bortezomib. As seen in the figure, the levels of BMP-2 increased upon treatment with 0.1 μM of MG132 inhibitor from approximately 1.50 a.u. in the non-treated sample to 5.95 a.u. in the treated sample. In the sample treated with 1 μM of MG132, the level of BMP-2 increased to approximately 9.51 a.u. Further, the levels of BMP-2 increased upon treatment with 0.1 μM of bortezomib inhibitor from approximately 1.50 a.u. in the non-treated sample to 6.55 a.u. in the treated sample. In the sample treated with 1 μM of bortezomib, the level of BMP-2 increased from approximately 1.50 a.u. to 9.68 a.u.

As may be appreciated, upon treatment with the above proteasome inhibitors, an increase in BMP-2 were exhibited in all but one example, where curucmin was dosed at 100 μM. It is noted that the error values are indicated on the graphs.

### Example 7

An examination of gene expression of BMP-1 was conducted using a number of proteasome inhibitors including, proteasome inhibitor-1, curcumin, MG-132 and bortezomib. Human neonatal dermal fibroblasts (AllCells, Catalog No. HN006001) were maintained in Dulbecco's modified Eagle's medium (DMEM) supplemented with 10% fetal bovine serum. Early passage cells were plated at 2×10⁴ cells/cm² and grown in humidified 5% CO₂ at 37° C. for 24 hours. Spent media was replaced the following day with assay media including Proteasome inhibitor-1 was added in DMEM at 1 μM and 0.1 μM. Untreated cells were used as the control group. For the human osteogenesis PCR array, the experimental group was treated only at 1 μM. In addition, in separate trials, spent media was replace by assay media of curcumin added in DMEM at 10 μM and 100 μM, MG-132 in DMEM at 0.1 μM and 1 μM, and bortezomib in DMEM at 0.1 μm and 1 μM.

Phenol-chloroform extraction was performed to isolate the total RNA for real time polymerase chain reaction array. More specifically, the total RNA was prepared using TRI REAGENT (Ambion, product number 9738), wherein the total RNA was obtained from the human neonatal dermal fibroblasts. Note that isolation was performed as per the instructions provided with the TRI REAGENT. The RNA was used for subsequent quantitative polymerase chain reaction (qPRC) gene expression of BMP₁ measured using hydrolysis probes (TAQMAN probes available from Applied Biosystems) as described further below.

Concentration and integrity of the obtained total RNA was assessed by absorbance in a spectrophotometer at 260 nm and 280 nm and by electrophoresis in 1% agarose with formaldehyde loading dye. For TAQMAN gene expression assays, cDNA was synthesized from 2 μg of total RNA using the High Capacity cDNA Reverse Transcription Kit (Applied Biosystems, product number 4368814), again following the protocol supplied with the kit. The resulting cDNA was diluted 1:10.

For TAQMAN gene expression, gene-specific fluorescent labeled TAQMAN primers for FAM-BMP1 (Applied Biosystems, product Hs0098677.g1) and TAQMAN reference VIC-18S ribosomal RNA (Applied Biosystems, product number 4310893E) were used to target the gene of interest. A cocktail of universal master mix (Applied Biosystems, product number 4369016) was prepared individually for both set of primers which contained Taq Polymerase, dNTPs, and buffer. Template cDNA's were added separately to respective individual wells in triplicate samples. PCR was performed on StepOne Plus Real Time System instrument (Applied Biosystems). The amplification program consisted of 1 cycle of 95° C. with 10 minute hold (hot start) followed by 50 cycles of 95° C. with 15 second annealing hold and 1 minute 60° C. specified acquisition hold.

For human osteogenisis PCR array, total RNA was prepared using silica matrix purification using RNEASY MINI KIT (Qiagen, product number 74101) according to the manufacturer's recommendations. cDNA was synthesized from 1 μg of purified DNA-free RNA accordingly to RT²First Strand Kit (SABiosciences, C-03). The resulting cDNA was diluted 1:30 and amplified. The procedure for the PCR array using real-time PCR is based on the RT² Profiler PCR array system instructions for Human Osteogenesis (SABiosciences, product number PAHS-026C). A cocktail reaction mix was setup as recommended by the PCR manufacturer (SABiosceiences, product number PA-012). PCR was performed using the same temperature parameters as used with the StepOne Plus Real Time System instrument. The Ct value of endogenous reference gene (i.e. 18s) was used to control for input RNA and then used to normalize target gene (i.e. BMP-1) tested from the same cDNA sample (Δ Ct), and then calibrated to an internal reference sample (ΔΔCt). Change in gene expression was determined by the expression 2̂-(ΔΔCt).

In addition, as alluded to above a similar methodology using 10 μmM and 100 μM of curcumin, 0.1 μM and 1 μM of bortezomib, and 0.1 μM and 1 μM of MG-132 proteasome inhibitors was performed.

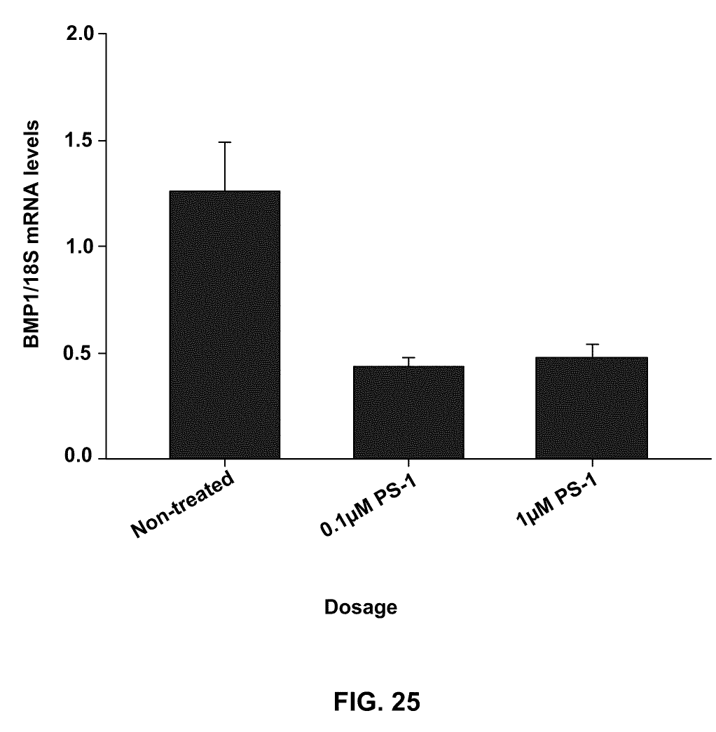

FIG. 25 illustrates BMP-1/18S mRNA levels in human dermal fibroblast cells after proteasome inhibitor treatment with 0.1 μM of PS-1 and 1 μm PS-1 as compared to the non-treated control. As can be seen, BMP-1 expression decreased upon treatment from approximately 1.26 a.u. seen in the nontreated sample to 0.436 a.u. in the sample treated with 0.1 μM of PS-1 and 0.480 a.u. in the sample treated with 1.0 μM of PS-1.

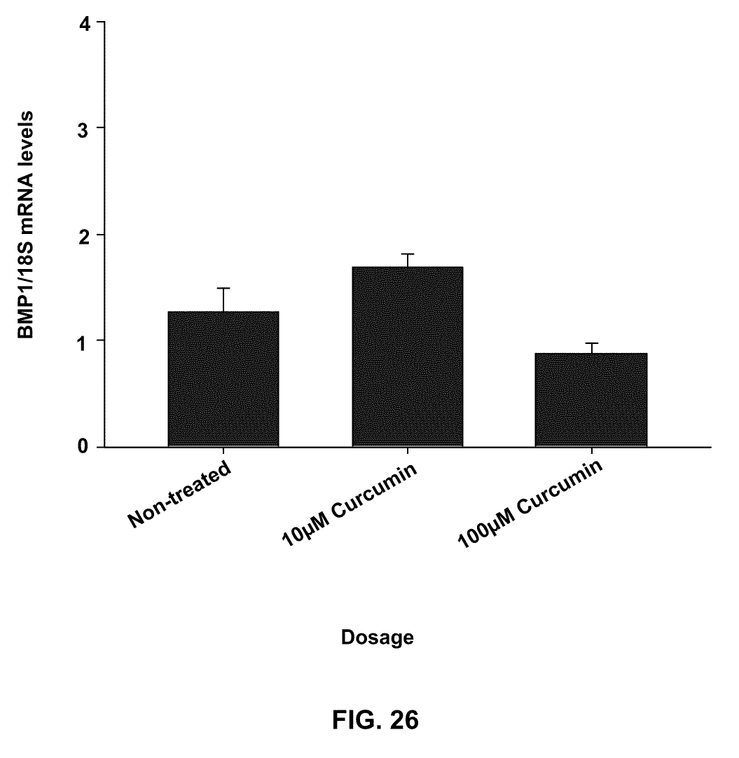

FIG. 26 illustrates BMP-1/18S mRNA levels in human dermal fibroblast cells after proteasome inhibitor treatment with 10 μM of curcumin and 100 μm of curcumin as compared to the non-treated control. As can be seen, BMP-1 expression increased upon treatment from approximately 1.26 a.u. seen in the nontreated sample to 1.68 a.u. in the sample treated with 10 μM of curcumin and 0.882 a.u. in the sample treated with 100 μM of curcumin.

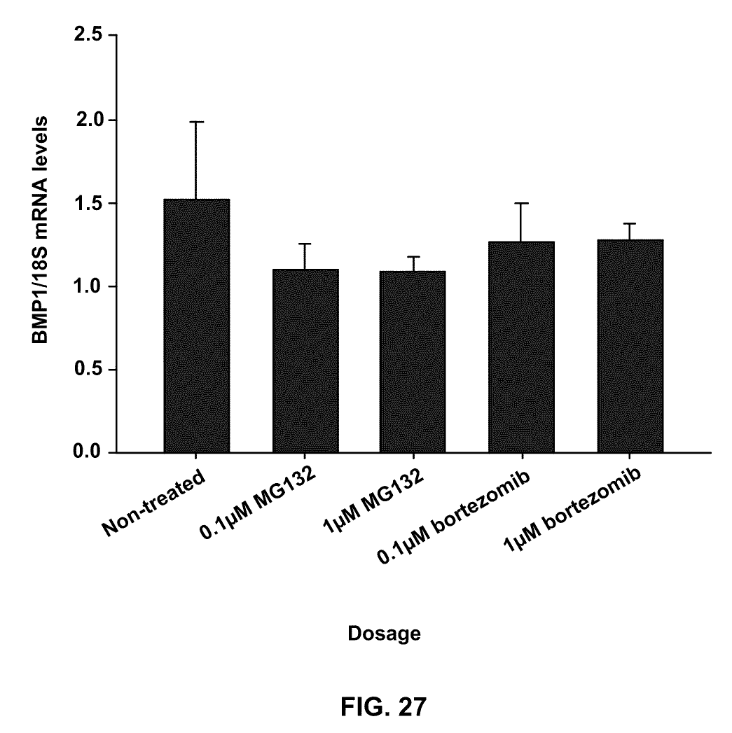

FIG. 27 illustrates BMP-1/18S mRNA levels in human dermal fibroblast cells after proteasome inhibitor treatment with 0.1 μM of MG132 and 1 μM of MG132 as compared to the non-treated control as well as treatment with 0.1 μM of bortezomib and 1 μM of bortezomib. As can be seen, BMP-1 expression decreased upon treatment with the MG132 from approximately 1.52 a.u. seen in the nontreated sample to 1.10 a.u. in the sample treated with 0.1 μM and to 1.09 a.u. in the sample treated with 1 μM. BMP-1 expression also decreased upon treatment with the bortezomib from approximately 1.50 a.u. seen in the nontreated sample to 1.27 a.u. in the sample treated with 0.1 μM and to 1.27 a.u. in the sample treated with 1 μM.

As can be seen from the above, addition of the proteasome inhibitors reduced BMP-1 levels in all but one example where 10 μM of curcumin was used to treat the sample. However, as can be seen in the graphs, improvements in the addition of MG132 and bortezomib may not be statistically significant.

### Example 8

An examination of gene expression of TFG-β₁ was conducted using a number of proteasome inhibitors including, proteasome inhibitor-1, curcumin, MG-132 and bortezomib. Human neonatal dermal fibroblasts (AllCells, Catalog No. HN006001) were maintained in Dulbecco's modified Eagle's medium (DMEM) supplemented with 10% fetal bovine serum. Early passage cells were plated at 2×10⁴ cells/cm² and grown in humidified 5% CO₂ at 37° C. for 24 hours. Spent media was replaced the following day with assay media including Proteasome inhibitor-1 was added in DMEM at 1 μM and 0.1 μM. Untreated cells were used as the control group. For the human osteogenesis PCR array, the experimental group was treated only at 1 μM. In addition, in separate trials, spent media was replace by assay media of curcumin added in DMEM at 10 μM and 100 μM, MG-132 in DMEM at 0.1 μM and 1 μM, and bortezomib in DMEM at 0.1 μm and 1 μM.

Phenol-chloroform extraction was performed to isolate the total RNA for real time polymerase chain reaction array. More specifically, the total RNA was prepared using TRI REAGENT (Ambion, product number 9738), wherein the total RNA was obtained from the human neonatal dermal fibroblasts. Note that isolation was performed as per the instructions provided with the TRI REAGENT. The RNA was used for subsequent quantitative polymerase chain reaction (qPRC) gene expression of TFG-β₁ measured using hydrolysis probes (TAQMAN probes available from Applied Biosystems) as described further below.

Concentration and integrity of the obtained total RNA was assessed by absorbance in a spectrophotometer at 260 nm and 280 nm and by electrophoresis in 1% agarose with formaldehyde loading dye. For TAQMAN gene expression assays, cDNA was synthesized from 2 μg of total RNA using the High Capacity cDNA Reverse Transcription Kit (Applied Biosystems, product number 4368814), again following the protocol supplied with the kit. The resulting cDNA was diluted 1:10.

For TAQMAN gene expression, gene-specific fluorescent labeled TAQMAN primers for FAM-TFG-β₁ (Applied Biosystems, product number Hs00998130.m1) and TAQMAN reference VIC-18S ribosomal RNA (Applied Biosystems, product number 4310893E) were used to target the gene of interest. A cocktail of universal master mix (Applied Biosystems, product number 4369016) was prepared individually for both set of primers which contained Taq Polymerase, dNTPs, and buffer. Template cDNA's were added separately to respective individual wells in triplicate samples. PCR was performed on StepOne Plus Real Time System instrument (Applied Biosystems). The amplification program consisted of 1 cycle of 95° C. with 10 minute hold (hot start) followed by 50 cycles of 95° C. with 15 second annealing hold and 1 minute 60° C. specified acquisition hold.

For human osteogenisis PCR array, total RNA was prepared using silica matrix purification using RNEASY MINI KIT (Qiagen, product number 74101) according to the manufacturer's recommendations. cDNA was synthesized from 1 μg of purified DNA-free RNA accordingly to RT²First Strand Kit (SABiosciences, C-03). The resulting cDNA was diluted 1:30 and amplified. The procedure for the PCR array using real-time PCR is based on the RT² Profiler PCR array system instructions for Human Osteogenesis (SABiosciences, product number PAHS-026C). A cocktail reaction mix was setup as recommended by the PCR manufacturer (SABiosceiences, product number PA-012). PCR was performed using the same temperature parameters as used with the StepOne Plus Real Time System instrument. The Ct value of endogenous reference gene (i.e. 18s) was used to control for input RNA and then used to normalize target gene (i.e. TFG-β₁) tested from the same cDNA sample (β Ct), and then calibrated to an internal reference sample (ββCt). Change in gene expression was determined by the expression 2̂-(ββCt).

In addition, as alluded to above a similar methodology using 10 μmM and 100 μM of curcumin, 0.1 μM and 1 μM of bortezomib, and 0.1 μM and 1 μM of MG-132 proteasome inhibitors was performed.

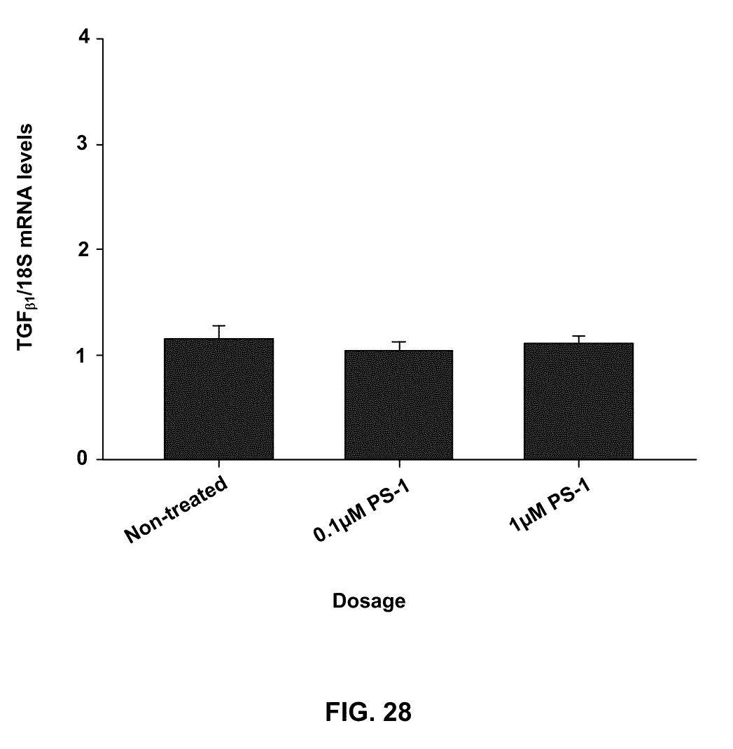

FIG. 28 illustrates the expression of TFG-β₁/18 mRNA levels in human dermal fibroblast cells after proteasome inhibitor treatment with 0.1 μM of PS-1 and 1 μm PS-1 as compared to the non-treated control. As can be seen, TFG-β₁ expression appear to slightly decrease upon treatment with proteasome inhibitor, remaining approximately between 1.15 a.u. seen in the nontreated sample to 1.03 a.u. in the sample treated with 0.1 μM of PS-1 and 1.11 a.u. in the sample treated with 1.0 μM of PS-1.

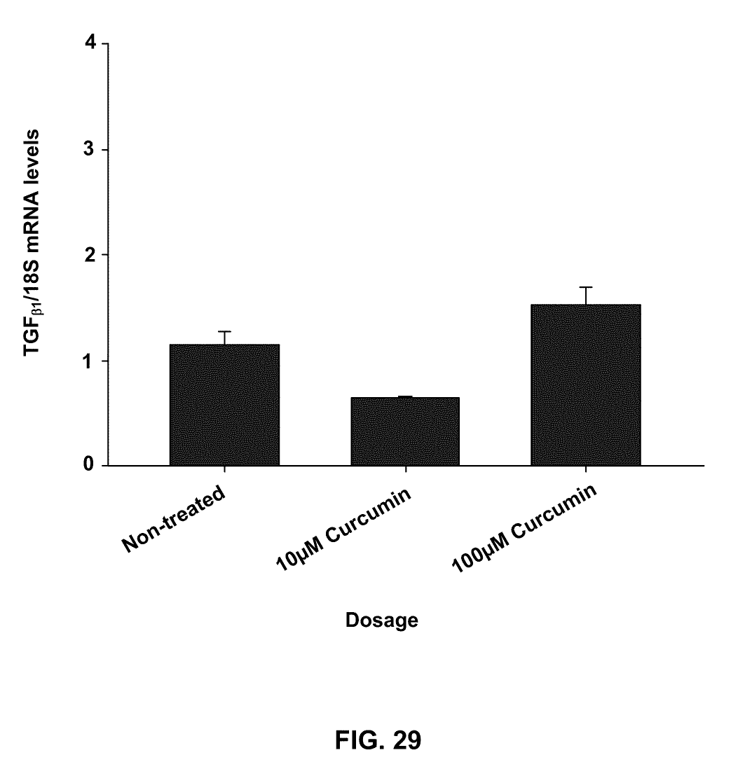

FIG. 29 illustrates the expression of TFG-β₁/18 mRNA levels in human dermal fibroblast cells after proteasome inhibitor treatment with 10 μM of curcumin and 100 μm of curcumin as compared to the non-treated control. As can be seen, TFG-β₁ expression appears to decrease with the addition of 10 μM of curcumin from 1.15 a.u. in the non-treated sample to 0.644 a.u. in the treated sample. Further, TGF-β₁ expression appears to increase with the addition of 100 μM of curcumin to 1.53 a.u.

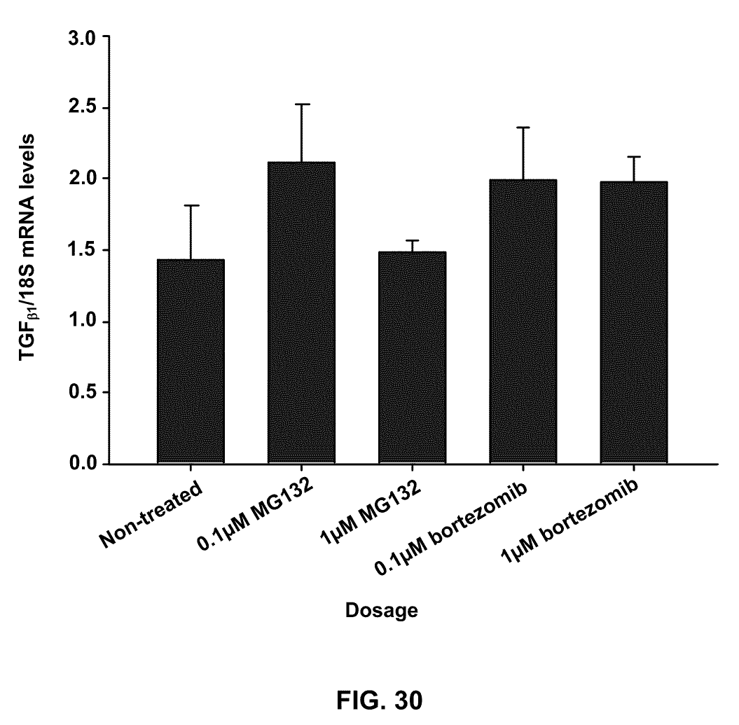

FIG. 30 illustrates the expression of TFG-β₁/18 mRNA levels in human dermal fibroblast cells after proteasome inhibitor treatment with 0.1 μM of MG132 and 1 μM of MG132 as compared to the non-treated control as well as treatment with 0.1 μM of bortezomib and 1 μM of bortezomib. As can be seen, TFG-β₁ expression appears to increase with the addition of 0.1 μM of MG132 from 1.50 a.u. in the non-treated sample to 2.12 a.u. TFG-β₁ expression appears to increase from 1.50 a.u. in the non-treated sample to 1.49 a.u. in the sample treated with the addition of 1 μM of MG132. Further, TFG-β₁ expression appears to increase with the addition of 0.1 μM of bortezomib from 1.43 a.u. in the non-treated sample to 1.99 a.u. A similar increase was also realized with the addition of 1 μM of bortezomib to 1.97 a.u.

As can be seen from the above, in all but three samples, an increase in TGF-β₁ expression was exhibited. In addition, of the three samples where a decrease was exhibited (both PS-1 samples and 10 μM of curcumin), the decrease exhibited in the PS-1 samples appear to be relatively statistically insignificant.

The foregoing description of several methods and embodiments has been presented for purposes of illustration. It is not intended to be exhaustive or to limit the claims to the precise steps and/or forms disclosed, and obviously many modifications and variations are possible in light of the above teaching. It is intended that the scope of the invention be defined by the claims appended hereto.

What is claimed is:

1. A method of treating wounded skin to control scarring, the method comprising:
administering an effective amount of a proteasome inhibitor to wounded skin of a subject.

2. The method of claim 1, wherein the proteasome inhibitor exhibits a half maximal inhibitory concentration (IC₅₀) for 20S proteasome in the range of 0.02 nM to 1,000 nM.

3. The method of claim 1, wherein the proteasome inhibitor comprises PS-1.

4. The method of claim 1, wherein the proteasome inhibitor comprises curcumin.

5. The method of claim 1, wherein the proteasome inhibitor comprises bortezomib.

6. The method of claim 1, wherein the proteasome inhibitor comprises MG-132.

7. The method of claim 1, wherein the composition is combined with a pharmaceutically acceptable carrier.

8. The method of claim 1, wherein wounded skin administered with said effective amount of said proteasome inhibitor exhibits a first tensile strength T₁ after 28 days and untreated wounded skin exhibits a second tensile strength T₂ after 28 days, wherein T₂*1.05≦T₁.

9. The method of claim 1, wherein wounded skin administered with said effective amount of said proteasome inhibitor exhibits a first thickness tᵢ after four days and untreated wounded skin exhibits a second thickness t₂ after four days, wherein t₂*1.05≦t₁.

10. The method of claim 1, wherein the subject is mammalian.

11. The method of claim 1, wherein the proteasome inhibitor is administered topically.

12. The method of claim 1, wherein administration is performed at intervals selected from the group consisting of one or more times a year, one or more times a month, one or more times a week and one or more times a day.

13. A method of increasing the tensile strength of wounded skin, the method comprising:
administering an effective amount of a proteasome inhibitor to wounded skin of a subject.

14. The method of claim 13, wherein the proteasome inhibitor exhibits a half maximal inhibitory concentration (IC₅₀) for 20S proteasome in the range of 0.02 nM to 1,000 nM.

15. The method of claim 13, wherein the proteasome inhibitor comprises PS-1.

16. The method of claim 13, wherein the proteasome inhibitor comprises curcumin.

17. The method of claim 13, wherein the proteasome inhibitor comprises bortezomib.

18. The method of claim 13, wherein the proteasome inhibitor comprises MG-132.

19. The method of claim 13, wherein the composition is combined with a pharmaceutically acceptable carrier.

20. The method of claim 13, wherein wounded skin administered with said effective amount of said proteasome inhibitor exhibits a first tensile strength T₁ after 28 days and untreated wounded skin exhibits a second tensile strength T₂ after 28 days, wherein T₂*1.05≦T₁.

21. The method of claim 13, wherein wounded skin administered with said effective amount of said proteasome inhibitor exhibits a first thickness tᵢ after four days and untreated wounded skin exhibits a second thickness t₂ after four days, wherein t₂*1.05≦t₁.

22. The method of claim 13, wherein the subject is mammalian.

23. The method of claim 13, wherein the proteasome inhibitor is administered topically.

24. The method of claim 13, wherein administration is performed at intervals selected from the group consisting of one or more times a year, one or more times a month, one or more times a week and one or more times a day.
